(19) United States
(12) Patent Application Publication
(10) Pub. No.: US 2026/0325579 A1
(43) Pub. Date: Oct. 22, 2026

(54) BIOMATERIAL FOR WOUND REGENERATIVE HEALING

(71) Applicant: The Regents of the University of California, Oakland, CA (US)

(72) Inventors: Sergei BUTENKO, Oakland, CA (US); Wendy LIU, Oakland, CA (US); Maksim PLIKUS, Oakland, CA (US)

(73) Assignee: The Regents of the University of California, Oakland, CA (US)

(21) Appl. No.: 19/104,666

(22) PCT Filed: Aug. 18, 2023
(86) PCT No.: PCT/US2023/072514

§ 371 Date: Feb. 18, 2025

Related U.S. Application Data
(60) Provisional application No. 63/399,579, filed on Aug. 19, 2022.

(51) Int. Cl. A61K 35/17; A61K 41/00; A61K 47/42; A61P 17/02

(52) U.S. Cl. CPC A61K 35/17; A61K 41/00; A61K 47/42; A61P 17/02

(57) ABSTRACT

The present embodiments include a biomaterial with immunomodulating properties engineered for complete incorporation into injured tissues while enhancing the regenerative healing activities of immune and stromal cells. The biomaterial may be used in, but not limited to, skin or muscle. A first component in some embodiments is gelatin or gelatin methacryloyl. In the present embodiments, this component consists of a specific composition and its modification with another therapeutic component. A second component of some embodiments, apoptotic neutrophil cells, has not previously been used in biomaterials for wound healing nor in combination with the first gelatin-based component.

### CROSS-REFERENCE TO RELATED APPLICATIONS

The present application claims priority to U.S. Provisional Patent Application No. 63/399,578 filed Aug. 19, 2022, the contents of which are incorporated herein by reference in their entirety.

### TECHNICAL FIELD

The present embodiments relate generally to medical devices and more particularly to methods and apparatuses for biomaterial engineering for wound healing.

### BACKGROUND

Two types of wound healing conditions present a great challenge and consume many health resources. The first is hard to heal chronic wounds such as diabetic foot ulcers, venous leg ulcers, and pressure ulcers (Frykberg, R. G. and J. Banks, Challenges in the Treatment of Chronic Wounds. Adv Wound Care (New Rochelle), 2015. 4(9): p. 560-582). Chronic wounds often occur in individuals with predispositions such as vascular disease or diabetes which lead to defects in the molecular and cellular pathways of the wound healing process. The second type is hyper fibrotic wounds that heal uncontrollably with excess scaring such as hypertrophic scars and keloids (Walmsley, G. G., et al., Scarless Wound Healing: Chasing the Holy Grail. Plastic and Reconstructive Surgery, 2015. 135(3): p. 907-917). The hyper fibrotic wounds are widely prevalent and often impose difficulties for postoperative recovery.

For chronic wounds, there are many treatments available however they require tremendous costs of manufacturing. These include recombinant growth factor proteins, engineered extracellular matrices (ECMs) and artificial skin, and negative pressure wound therapy (NPWT). Using such technologies, treatment of ulcers lasts on average 12 to 13 months and recur in up to 60% to 70% of patients. For the second type of challenging wound, hyper fibrotic wounds, there are very few treatment options. Small lesions of this type can be treated with steroids or cryotherapy with limited success. However, for large lesions, the only successful technique includes surgical excision combined with chemotherapy and/or radiotherapy (Limmer, E. E. and D. A. Glass, 2nd, A Review of Current Keloid Management: Mainstay Monotherapies and Emerging Approaches. Dermatol Ther (Heidelb), 2020. 10(5): p. 931-948). In this type of treatment, the surgical excision initiates a healing process that is moderated by chemotherapy, phototherapy, or radiotherapy. This type of healing lasts on average 5 to 6 months but it depends on the size of the excision and sometimes lasts for much longer. This treatment requires expensive radiology or laser devices and an operator. Also, this treatment is susceptible to recurrences. Importantly, hyper fibrotic wounds might occur following surgeries. Therefore, affordable post operative management with biomaterials can improve wound healing that will limit fibrotic wounds in the first place.

It is against this technological backdrop that the present Applicant sought a technological solution to these and other problems rooted in this technology.

### SUMMARY

The present embodiments relate to a biomaterial with immunomodulating properties engineered for complete incorporation into injured tissues while enhancing the regenerative healing activities of immune and stromal cells. The biomaterial is suitable for the skin but is suitable for other tissues such as muscle. In a murine model of skin wounds, the biomaterial improves healing by stimulating the infiltration of immune cells and activating them in a way that inhibits inflammation and promotes tissue repair. This results in enhanced production of blood vessels which can be used to improve chronic wounds. Additionally, this results in the production of growth factors that can be used to improve hyper fibrotic scarring. The base for this biomaterial in some embodiments is a gelatin scaffold that is a form of processed animal collagen. Gelatin is a biocompatible biomaterial and is FDA approved (Naomi, R., et al., Natural-Based Biomaterial for Skin Wound Healing (Gelatin vs. Collagen): Expert Review. Polymers (Basel), 2021. 13(14)). The gelatin component is supplemented with methacrylic anhydride-forming a cross-linkable biomaterial-gelatin methacryloyl (GelMA). Furthermore, supplementing GelMA with a photoinitiator allows tunable GelMA polymerization in wounds using UV light. The degree of crosslinking and hence final physical features of GelMA are tunable by the amount of UV exposure. The GelMA scaffold of some embodiments is 10% of GelMA and 90% physiological solution. This hydrogel provides key features to improve healing; isolation and hydrated environment, diffusion of soluble molecular signals, cellular migration, and finally absorption of all its components into tissue. In some embodiments, the GelMA part is mixed with apoptotic neutrophil cells (AN) and forms GelMA-AN. The AN are produced from an allogeneic source in the lab (mouse to mouse) but can be potentially produced from autologous blood in humans (self to self). AN provides immunomodulating components that act on central immune coordinators-macrophage cells. AN uptake by macrophages programs them to an anti-inflammatory and pro-repair activation. The antagonizing effect of apoptotic cells on inflammation has been recently studied intensively. Importantly, AN implantation is safe and approved for the treatment of hyperinflammatory conditions (Karbian, N., et al., Apoptotic cell therapy for cytokine storm associated with acute severe sepsis. Cell Death Dis, 2020. 11(7): p. 535; Toussirot, E., et al., Mini-Review: The Administration of Apoptotic Cells for Treating Rheumatoid Arthritis: Current Knowledge and Clinical Perspectives. Front Immunol, 2021. 12: p. 630170). Macrophages infiltrating into GelMA-AN encounter a unique AN component that stimulates their production of growth and angiogenic factors. This results in the repair of skin with increased blood vasculature.

Briefly, the pro-healing mechanism of the GelMA-AN of some embodiments works like the following. Both components-GelMA and AN interact with the wounded tissue. The central beneficial effect is through the interaction of infiltrating macrophage cells with GelMA and AN. Macrophages uptake GelMA and in response produce anti-inflammatory and pro-healing signals. Similarly, macrophages also uptake AN and in response produce anti-inflammatory and pro-healing signals. Importantly, both components induce different profiles of healing signals that together can work synergistically. Furthermore, the inclusion of the AN component inhibits the degradation of GelMA hence allowing a prolonged effect of GelMA-AN in the healing tissue. This results in prolonged exposure of stromal cells such as fibroblasts and vascular endothelium to growth and angiogenic factors and thus enhances the formation of blood vessels and extracellular matrix in healed tissue.

### BRIEF DESCRIPTION OF THE DRAWINGS

These and other aspects and features of the present embodiments will become apparent to those ordinarily skilled in the art upon review of the following description of specific embodiments in conjunction with the accompanying figures, wherein:

FIG. 1A is a schematic illustration of example GelMA-AN fabrication according to embodiments.

FIG. 1B illustrates example aspects of a GelMA-AN mechanism of action for immunomodulating skin wound healing and promoting tissue regeneration according to embodiments.

FIGS. 2A to 2E illustrate example aspects of how GelMA-AN enhances angiogenesis and immune infiltrate on day 14 post wounding compared to sham and GelMA-only wounds according to embodiments.

FIGS. 3A and 3B illustrate example aspects of how Macrophages are stimulated by the GelMA to secrete inflammatory signaling which is modified by AN towards pro-healing signaling according to embodiments.

FIG. 4 is an example diagram that illustrates that Macrophages are stimulated by the GelMA-AN combination provides a complementary production of key growth factors that drive angiogenesis and extracellular matrix repair according to embodiments.

FIGS. 5A to 5C illustrate example aspects of how Macrophages uptake GelMA and process it in phagolysosomes according to embodiments.

FIG. 6 is an example diagram that illustrates that Macrophages are modulated by GelMA to reduce inflammation and increase anti-inflammatory cytokines according to embodiments.

FIGS. 7A and 7B illustrate example aspects of how on post wounding day 5, Soft GelMA is infiltrated and degraded to be remodeled into the nascent dermis according to embodiments.

FIGS. 8A and 8B illustrate example aspects of how on post wounding day 10, Soft GelMA is infiltrated and degraded to be remodeled into the nascent dermis according to embodiments.

FIG. 9 illustrate example aspects of how scarring is reduced by GelMA in a stiffness-dependent manner according to embodiments.

FIGS. 10A and 10B illustrate example aspects of how stiff GelMA induces more inflammatory activity in key immune cells during wound healing according to embodiments.

FIG. 11 illustrates example aspects of how stiff GelMA stimulates macrophage mechanotransduction according to embodiments.

FIGS. 12A to 12I illustrate example aspects of how GelMA increased stiffness potentiates macrophage response to LPS by elevating the production of ROS, mitochondrial fission, and glycolysis according to embodiments.

FIG. 13 is a diagram illustrating example aspects of specific aims in accordance with embodiments.

FIG. 14 is a diagram illustrating example aspects of the role of immune cells and angiogenesis wound healing.

FIGS. 15A to 15C illustrate example Angiogenic potential of GelMA-AN according to embodiments.

FIGS. 16A to 16C illustrate example synergistic effects of GelMA-AN on Mφ polarization and angiogenesis according to embodiments.

FIGS. 17A to 17C illustrate example GelMA crosslinking modulates Mφ phenotype and scarring of wounds according to embodiments.

FIG. 18 illustrates example Mo uptake of labeled AN according to embodiments.

FIG. 19 illustrates example Crosslinking influences GelMA uptake by Mos according to embodiments.

FIG. 20 illustrates example Tie1/2GFP imaged at 15 d post wounding according to embodiments.

FIG. 21 illustrates example spatial transcriptomics of murine 13 d skin wounds according to embodiments.

### DETAILED DESCRIPTION

The present embodiments will now be described in detail with reference to the drawings, which are provided as illustrative examples of the embodiments so as to enable those skilled in the art to practice the embodiments and alternatives apparent to those skilled in the art. Notably, the figures and examples below are not meant to limit the scone of the present embodiments to a single embodiment, but other embodiments are possible by way of interchange of some or all of the described or illustrated elements. Moreover, where certain elements of the present embodiments can be partially or fully implemented using known components, only those portions of such known components that are necessary for an understanding of the present embodiments will be described, and detailed descriptions of other portions of such known components will be omitted so as not to obscure the present embodiments. Embodiments described as being implemented in software should not be limited thereto, but can include embodiments implemented in hardware, or combinations of software and hardware, and vice-versa, as will be apparent to those skilled in the art, unless otherwise specified herein. In the present specification, an embodiment showing a singular component should not be considered limiting; rather, the present disclosure is intended to encompass other embodiments including a plurality of the same component, and vice-versa, unless explicitly stated otherwise herein. Moreover, applicants do not intend for any term in the specification or claims to be ascribed an uncommon or special meaning unless explicitly set forth as such. Further, the present embodiments encompass present and future known equivalents to the known components referred to herein by way of illustration.

According to certain general aspects, the present embodiments relate to a biomaterial based on a gelatin (Gel) scaffold that is a product of denatured animal collagen (Id.). The Gel is supplemented with Methacrylic Anhydride (MA) (CAS #: 760-93-0) to provide functional groups for covalent crosslinking-forming GelMA (Loessner, D., et al., Functionalization, preparation and use of cell-laden gelatin methacryloyl-based hydrogels as modular tissue culture platforms. Nat Protoc, 2016. 11(4): p. 727-46). The GelMA scaffold is mixed with immunomodulating Apoptotic Neutrophil cells (AN), thus forming GelMA-AN. By the addition of a photoinitiator Irgacure-2959 (CAS #: 106797-53-9), GelMA-AN crosslinking can be catalyzed by exposure to UV light.

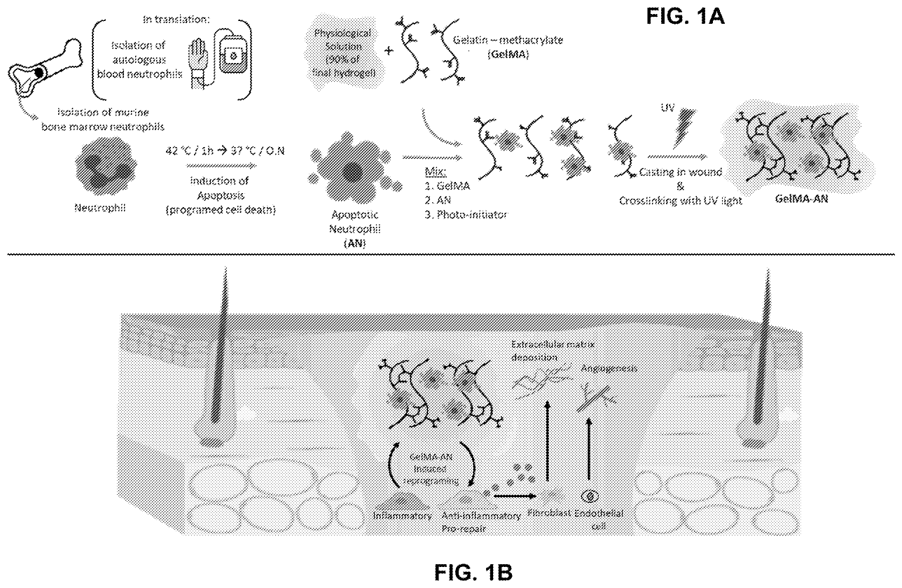

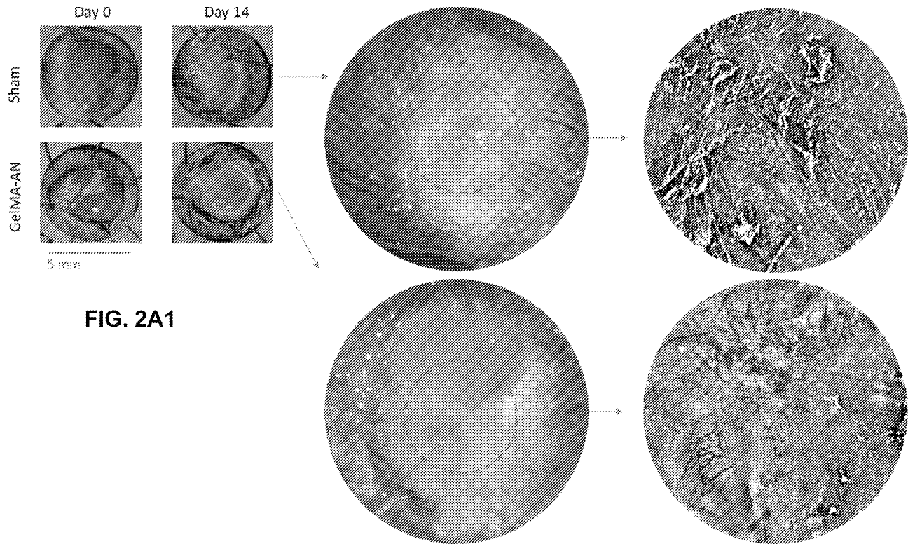

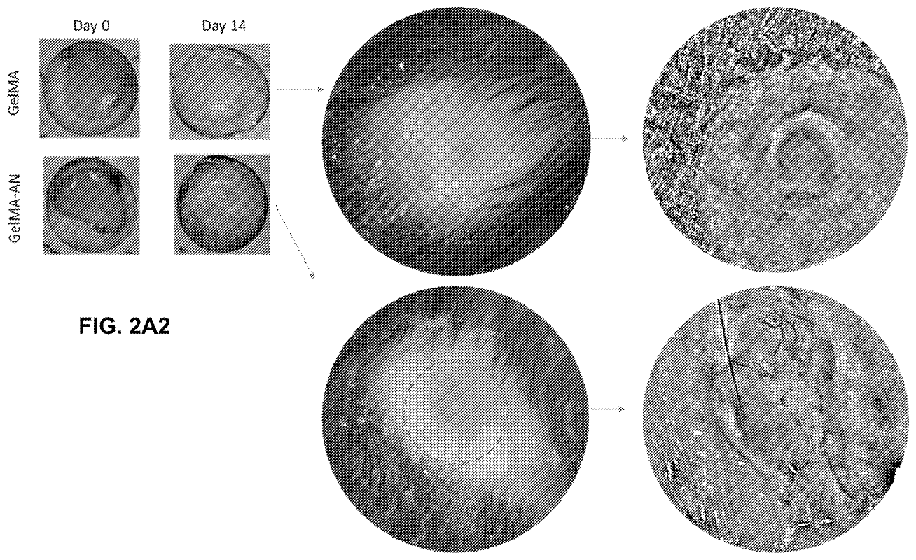

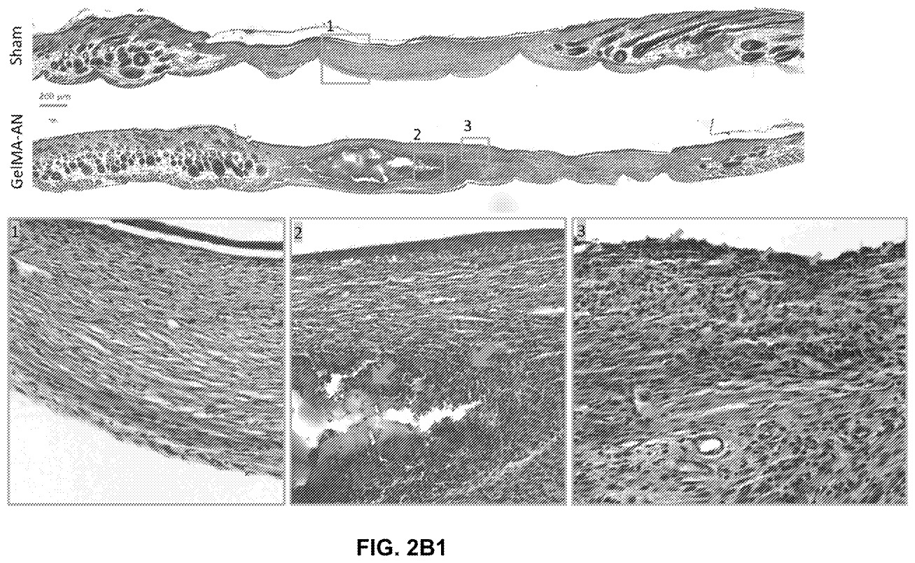

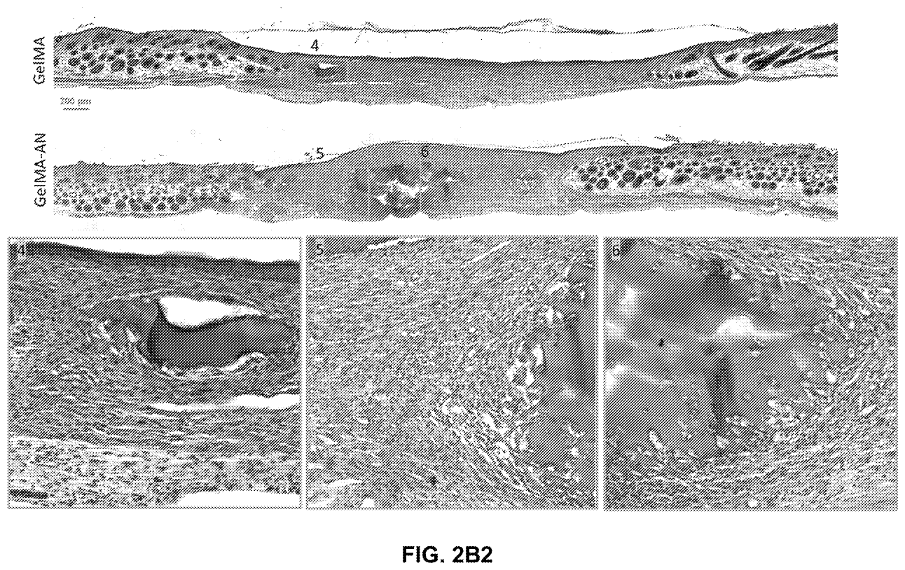

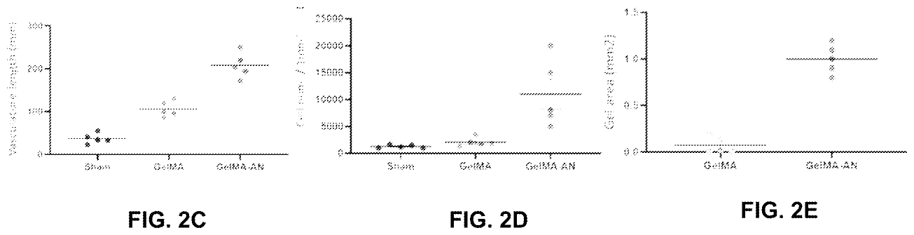

GelMA-AN is thus a product of two components (Gel and AN) whose individual immunomodulating properties are being uncovered in recent years (Id.) GelMA-AN is a novel method to deliver immunomodulating AN into wounds. Together they achieve a synergistic effect of combined Gel and AN to promote regenerative wound healing. In-vivo and in-vitro experiments show that the mechanism of GelMA-AN acts in wounds by modulating key immunity and wound healing orchestrators-macrophage cells. They are modulated in a way that promotes the resolution of inflammation in wounds and the renair activity of stromal cells (e.g. FIGS. 1A and 1B). GelMA-AN shows complete incorporation into healing tissue of skin wounds while promoting immune infiltration and enhancing the healing activities of macrophages to stimulate stromal cell production of extracellular matrix and enhanced angiogenesis (e.g. FIGS. 1A and 1B). Mouse wounds treated with GelMA-AN show enhanced angiogenesis and immune infiltration (e.g. FIGS. 2A-2E described below). With these features that are missing in chronic non-healing wounds (Id.), GelMA-AN holds the potential to achieve their healing. Additionally, GelMA-AN provides a physical scaffold that also drives the production of growth factors, two features that combined hold the potential to ameliorate hyper fibrotic scarring following surgical wounds (Abdelhakim, M., X. Lin, and R. Ogawa, The Japanese Experience with Basic Fibroblast Growth Factor in Cutaneous Wound Management and Scar Prevention: A Systematic Review of Clinical and Biological Aspects. Dermatol Ther (Heidelb), 2020. 10(4): p. 569-587; Wei, Y., et al., The clinical effectiveness and safety of using epidermal growth factor, fibroblast growth factor and granulocyte-macrophage colony stimulating factor as therapeutics in acute skin wound healing: a systematic review and meta-analysis. Burns Trauma, 2022. 10: p. tkac002).

Gelatin-based scaffolds have been documented to promote angiogenesis in the literature. The mechanism of GelMA-AN operates through the specific combination of GelMA with AN that prolongs the Gel degradation process compared to GelMA alone, thus prolonging the pro-healing effect and enhancing wound angiogenesis (e.g. FIGS. 2A-2E). GelMA is found to be a highly suitable vehicle for AN delivery based on its structural and immunomodulating properties which are detailed in the following. Gel is a denatured form of collagen that can be prepared readily and economically from an animal source. Gel shares the high biocompatibility of natural collagen, and its clinical usage is FDA approved. In its collagen-denatured form, Gel lacks the collagen-inherent ability to form fibrils thereby exposing the integrin-binding domain (RGD) which is constrained in the collagen precursor form (Davidenko, N., et al., Evaluation of cell binding to collagen and gelatin: a study of the effect of 2D and 3D architecture and surface chemistry. J Mater Sci Mater Med, 2016. 27(10): p. 148). These provide anchors for infiltrating cells in a healing wound. Furthermore, Gel exposes proteolytic cleavage sites that allow degradation by matrix metalloproteases more readily than collagen (Laronha, H. and J. Caldeira, Structure and Function of Human Matrix Metalloproteinases. Cells, 2020. 9(5)). By these two features, the Gel scaffold is gradually remodeled into a nascent healed skin. The denatured form of Gel makes it a highly modifiable precursor to form covalently crosslinked scaffolds (C. Echave, M., et al., Gelatin as biomaterial for tissue engineering. Current pharmaceutical design, 2017. 23(24): p. 3567-3584). To modify Gel, embodiments combine MA residues to form GelMA that can be crosslinked. These and other embodiments use a highly hydrated scaffold (10% GelMA/ 90% water) that enables an isolated and hydrated environment that serves as a buffer from the external environment and a platform for the infiltration of immune cells and their migration into the wound. In addition to the structural properties, it is found that GelMA provides inherent immunomodulatory properties when interacting with macrophages.

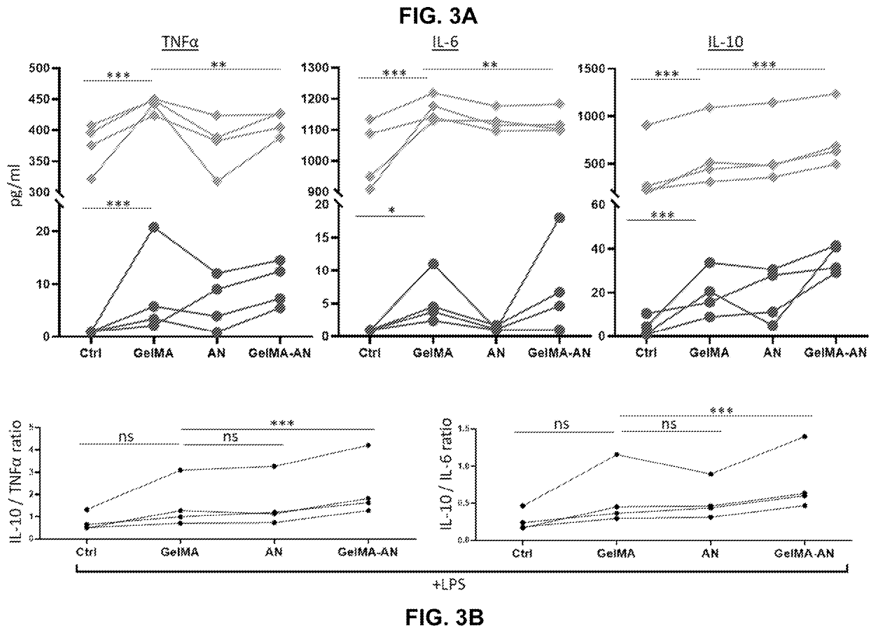

GelMA stimulates macrophage secretion of key inflammatory cytokines TNFα, IL-6 and IL-10 under baseline and further potentiates macrophage activation in response to the bacterial mimetic lipopolysaccharide (LPS) by increasing secretion of these cytokines compared to macrophages without GelMA (see FIG. 3A). Addition of AN to GelMA reduces secretion of key inflammatory cytokines TNFa, IL-6 while enhancing pro-healing IL-10. This shows that GelMA alone stimulates macrophage activity which corresponds to increased inflammatory infiltrate observed in wounds, however the effect of adding AN shifts the balance between IL-10 to TNFα or IL-6, indicating to skew macrophages towards pro-healing signaling (see FIG. 3B).

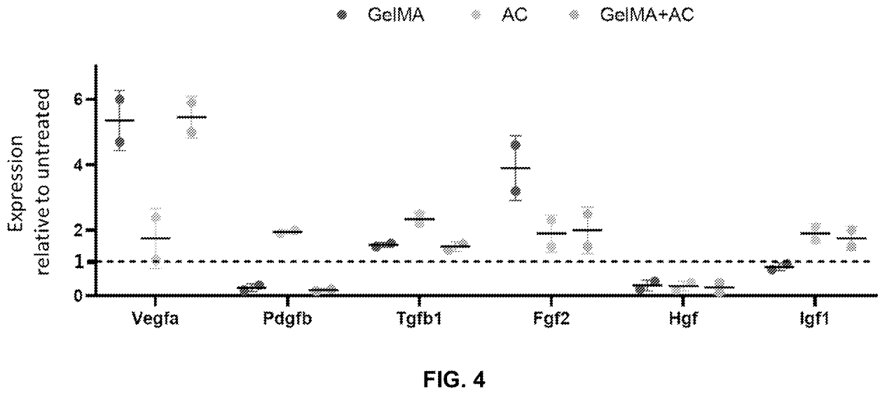

GelMA drives macrophages to a highly beneficial activity for wound healing by stimulating the production of central anti-inflammatory and pro-healing signals such as growth factors TGFβ, VEGFA, and FGF2 (see FIG. 4). The second immunomodulatory component-AN is also engulfed and processed by macrophages and stimulates TGFB, PDGF, FGF2, and IGF. A GelMA-AN combination provides a complementary production of growth factors while also prolonging GelMA scaffolds in wounds (see FIG. 2E) thereby enhancing the effect on healing. This is likely to be a result of an increased phagocytic burden on the macrophages that delays the degradation of hydrogels.

FIGS. 2A to 2I illustrate example aspects of how GelMA-AN enhances angiogenesis and immune infiltrate on day 14 post wounding compared to sham and GelMA-only wounds according to embodiments. FIG. 2A1 and 2A2 illustrate a pair of wounds on each mouse was created and each wound was treated with either GelMA or GelMA-AN or left untreated (sham). Top: sham vs. GelMA-AN; bottom: GelMA vs. GelMA-AN. 14 days post wounding the wounds were harvested and imaged. Left: images of day 1 vs. day 14; Middle: day 14 wounds; Right: close up on wound center images with enhanced contrast FIG. 2B1 and 2B2 illustrate H&E stains. Top: sham vs. GelMA-AN; bottom: GelMA vs. GelMA-AN. Squares: 1: Dermis in sham wounds; 2: GelMA-AN, blue arrows indicate dense immune infiltrate with penetration into GelMA-AN; 3: GelMA-AN, blue arrows indicate a large number of blood vessels including in unusual location proximal to the epidermis; 4. Small remains of GelMA; 5. GelMA-AN, blue arrows indicate a large number of blood vessels proximal to GelMA-AN; 6. dense immune infiltrate with penetration into GelMA-AN. FIG. 2C illustrates that Vasculature length is significantly longer in GelMA-AN compared to sham and GelMA. FIG. 2D illustrates that Cell numbers are significantly higher in GelMA-AN compared to sham and GelMA. FIG. 2E illustrates that Gel area is significantly larger in GelMA-AN compared to GelMA.

FIGS. 3A and 3B illustrate example aspects of how Macrophages are stimulated by the GelMA to secrete inflammatory signaling which is modified by AN towards pro-healing signaling according to embodiments. FIG. 3A illustrates that GelMA significantly increase secretion of key inflammatory cytokines TNFα, IL-6 an IL-10. In response to LPS stimulation, GelMA further enhance secretion of these cytokines compared to control. Addition of AN to GelMA reduces secretion of key inflammatory cytokines TNFα, IL-6 while enhancing pro-healing IL-10. FIG. 3B illustrates that the addition of AN to GelMA shifts the balance between IL-10 to TNFα or IL-6, indicating to skew macrophages to pro-healing signaling compared to macrophage response to GelMA only.

FIG. 4 illustrates example aspects of how Macrophages stimulated by the GelMA-AN combination provides a complementary production of key growth factors that drive angiogenesis and extracellular matrix repair according to embodiments. BMDMs seeded with GelMA, AN, or combined for 24h. Then, harvested and gene expression was analyzed by qPCR.

Macrophages engulf AN in a non-inflammatory process termed efferocytosis (Cotter, T., A. M. deCathelineau, and P. M. Henson, The final step in programmed cell death: phagocytes carry apoptotic cells to the grave. Essays in biochemistry, 2003. 39: p. 105-117). This clearance is a critical checkpoint to successful wound healing and triggers an anti-inflammatory and pro-resolution transition, which blocks excessive neutrophil recruitment and eliminates early inflammatory components (Doran, A. C., A. Yurdagul, and I. Tabas, Efferocytosis in health and disease. Nature Reviews Immunology, 2020. 20(4): p. 254-267). Defects in this stage often give rise to non-healing wounds (Phillipson, M. and P Kubes The Healing Power of Neutrophils. Trends in Immunology, 2019. 40(7): p. 635-647). During the resolution of inflammation, macrophages undergo a phenotype conversion to anti-inflammatory macrophages, which are characterized by the production of anti-inflammatory cytokines such as IL-10 and interferon-beta (IFN-β) (Satyanarayanan, S. K., et al., IFN-β is a macrophage-derived effector cytokine facilitating the resolution of bacterial inflammation. Nature Communications, 2019. 10(1): p. 1-16; Butenko, S., et al., ACKR2 limits skin fibrosis and hair loss through IFN-β. The FASEB Journal, 2021. 35(10)). The AN additive provides a novel anti-inflammatory and pro-healing component that is intensively studied currently as a treatment for hyper inflammatory conditions (Dangi, A., S. Yu, and X. Luo, Apoptotic cell-based therapies for promoting transplantation tolerance. Curr Opin Organ Transplant, 2018. 23(5): p. 552-558; Saas, P., et al., Harnessing Apoptotic Cell Clearance to Treat Autoimmune Arthritis. Front Immunol, 2017. 8: p. 1191). The AN are completely biocompatible both in lab and in clinic. In mice, the AN are transplanted in an allogeneic method. However, in humans, this can potentially be developed in an autologous method. Human neutrophils can be readily obtained from a patient's blood and then similarly rendered apoptotic and mixed with GelMA. AN stimulate macrophage reprograming during inflammation and skew them to anti-inflammatory and pro-healing activity (Elliott, M. R., K. M. Koster, and P. S. Murphy, Efferocytosis signaling in the regulation of macrophage inflammatory responses. The Journal of Immunology, 2017. 198(4): p. 1387-1394). This is likely to occur during wound healing thus the AN enhance the anti-inflammatory and pro-healing effect of GelMA-AN hydrogels. Macrophage anti-inflammatory activation is closely tied to pro-repair activation that is characterized by the production of reparative growth factors such as transforming growth factor-beta (TGF-β), vascular endothelial growth factor (VEGF), and platelet-derived growth factor (PDGF) (Wynn, T. A. and K. M. Vannella, Macrophages in Tissue Repair, Regeneration, and Fibrosis. Immunity, 2016. 44(3): p. 450-462). Interestingly, in the treatment of chronic wounds, there can be found usage of growth factors such as FGF and EGF (Krzyszczyk, P., et al., The Role of Macrophages in Acute and Chronic Wound Healing and Interventions to Promote Pro-wound Healing Phenotypes. Front Physiol, 2018. 9: p. 419). Also, EGF and FGF2 together with several other growth factors, including PDGF, VEGF, and TGF-β, have demonstrated efficacy for surgical use to enhance healing and reduce scarring. (Park, J. W., S. R. Hwang, and I. S. Yoon, Advanced Growth Factor Delivery Systems in Wound Management and Skin Regeneration. Molecules, 2017. 22(8)·Trengove N. J., H. Bielefeldt-Ohmann, and M. C. Stacey, Mitogenic activity and cytokine levels in non-healing and healing chronic leg ulcers. Wound Repair Regen, 2000. 8(1): p. 13-25). Importantly, GelMA-AN can provide the same growth and angiogenic factors by stimulating their production by endogenous macrophages (see FIG. 4). Importantly, AN additive prolongs GelMA-AN persistence in wounds and thus prolongs the pro-angiogenic effect of the hydrogels that results in increased vasculature (e.g. FIGS. 2A-2E). Further comparative analysis is provided below.

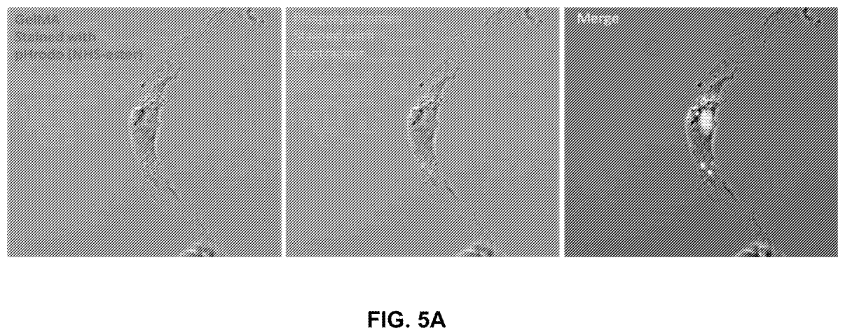

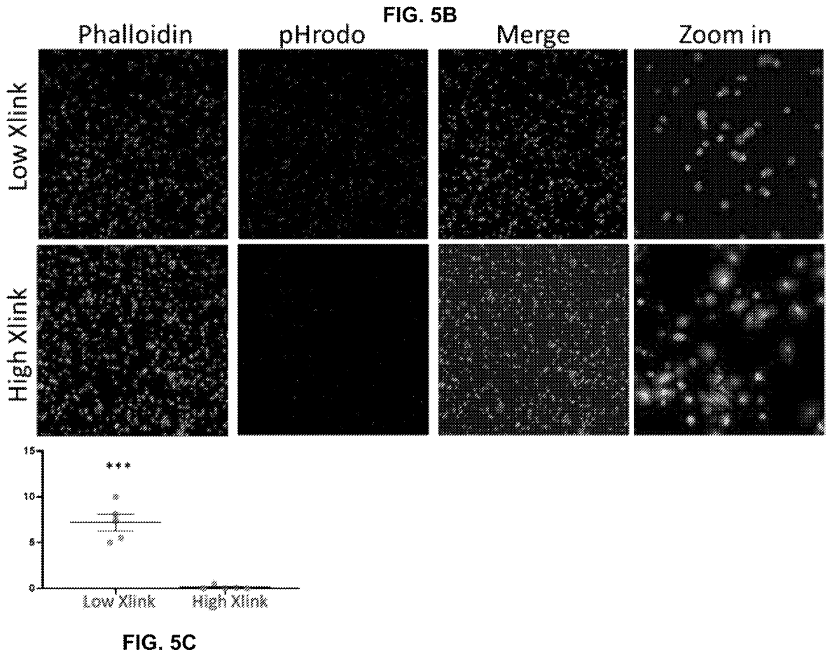

GelMA is readily taken up by macrophages (e.g. FIGS. 5A-5C). The degree of GelMA crosslinking affects its accessibility and is inversely related to its for phagocytosis by macrophages. Lower crosslinking in GelMA that is polymerized by 1 minute of UV exposure is taken up by macrophages significantly more than higher crosslinked—5 minutes UV exposure (see. FIG. 5C). Of note, when macrophages are cultured on 1-minute GelMA, it directs them to the highly beneficial activity for wound healing that is shown in FIG. 4.

FIGS. 5A to 5C illustrate example aspects of how macrophages uptake GelMA and process it in phagolysosomes according to embodiments. FIG. 5A illustrates BMDMs were seeded for 4 h. on GelMA which was supplemented with a fluorescent probe that signals in an acidic environment (red). Additionally, BMDMs were stained with lysosome homing dye (green) and nuclear dye (blue). FIG. 5B illustrates BMDMs were seeded on GelMA with a low or high degree of crosslinking that was regulated by the amount of UV exposure—1 or 5 minutes, respectively. FIG. 5C illustrates that the lower crosslinking allows a more accessible substrate which was phagocytosed significantly more by macrophages.

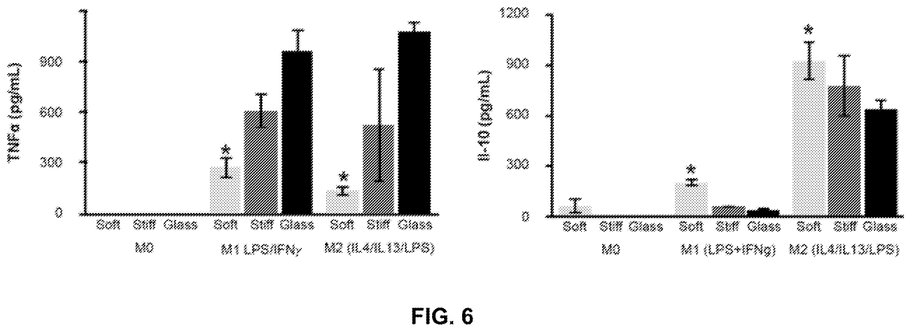

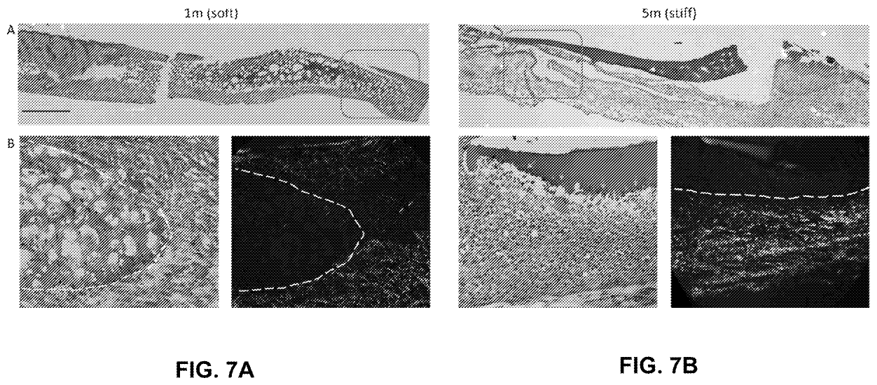

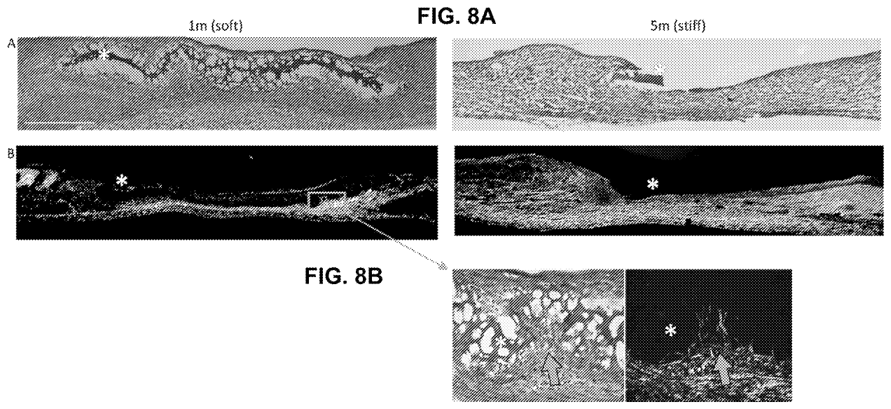

The degree of GelMA crosslinking effects also changes the stiffness of the material. Immune cells are sensitive to their physical environment and respond to mechanical stimulation. Mechanical cues are converted into biochemical signals through the process of mechanotransduction and regulate immunity. These pathways regulate immune cell activities in wound healing and response to biomaterials. Therefore, GelMA scaffold are studied through the lens of mechanoimmunology concerning the question of what mechanical properties will produce a favorable immune response and biomaterial-guided wound healing. GelMA crosslinking by UV light enables in situ tunable polymerization. The effect of UV exposure duration on induced crosslinking in GelMA and the resulted properties and biocompatibility are studied. It is found that one minute (1 m) of UV results in a biocompatible GelMA compared to five minutes (5 m). The 5 m GelMA has a 150 kDa stiffens and inflicts a stronger immune response in comparison to the 1 m GelMA which has a 3 kDa stiffness. Examining macrophages in-vitro effect 1 m vs. 5 m GelMA showed that macrophage response to inflammatory stimulus while seeded on 1 m produces less inflammatory activation. 5 m stiffer GelMA enhances macrophage pro-inflammatory activation in response to LPS (see FIG. 6). To test the mechanical properties of the GelMA in-vivo, applied were 1 m and 5m gels on murine 5 mm punch wounds. It was found that at post wounding day five (D5) and ten (D10), the Im GelMA was highly penetrable for cells compared to 5 m which does not allow cell infiltration (see FIGS. 7A and 7B). Additionally, the 1 m was incorporated into the skin and degraded faster at D5 and D10, compared to 5 m which by D10 couldn't incorporate and was ejected out of the skin (see FIGS. 8A and 8B).

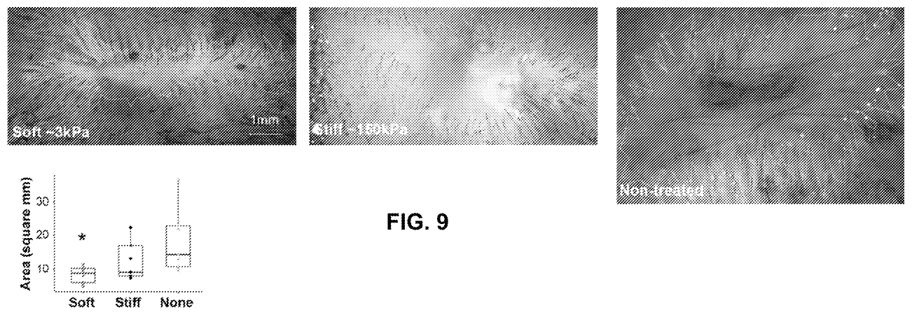

Eventually, 1 m results in less scarring compared to 5 m GelMA or wounds covered with an adhesive dressing (Tegaderm) without GelMA (see FIG. 9).

FIG. 6 illustrates aspects of how macrophages are modulated by GelMA to reduce inflammation and increase anti-inflammatory cytokines according to embodiments.

FIGS. 7A and 7B illustrate example aspects of how on post wounding day 5, Soft GelMA is infiltrated and degraded to be remodeled into the nascent dermis according to embodiments. FIG. 7A illustrates H&E staining of PWD 5 wound sections indicating reepithelization above soft GelMA (left). Without reepithelization in stiff GelMA. Scale 500 μm. FIG. 7B illustrates PSR & hematoxylin staining from PWD 5 indicating cell infiltration of GelMA (soft), cell alignment on GelMA edge (stiff), and polarized light imaging of PSR stained collagen indicating the absence of collagen in GelMA at PWD 5. Yellow line Dermis-GelMA border. Green arrows—cell alignment on stiff GelMA border.

FIGS. 8A and 8B illustrate example aspects of how on post wounding day 10, Soft GelMA is infiltrated and degraded to be remodeled into the nascent dermis according to embodiments. FIG. 8A shows H&E staining of PWD 10 wound sections indicating reepithelization above soft GelMA with degradation of GelMA and reepithelization below stiff GelMA with ejection. Scale 500 um. FIG. 8B shows Polarized light imaging of PSR stained collagen indicating collagenous dermis and matured collagen boundless deposition into digested regions of soft GelMA. Yellow asterisk—GelMA. Green arrow—matured collagen boundless in soft GelMA.

FIG. 9 illustrates example aspects of how scarring is reduced by GelMA in a stiffness-dependent manner according to embodiments. As shown, on day 28 post wounding the area of the scar is reduced in GelMA treated wounds, compared to non-treated. Specifically, soft GelMA significantly reduces scar area.

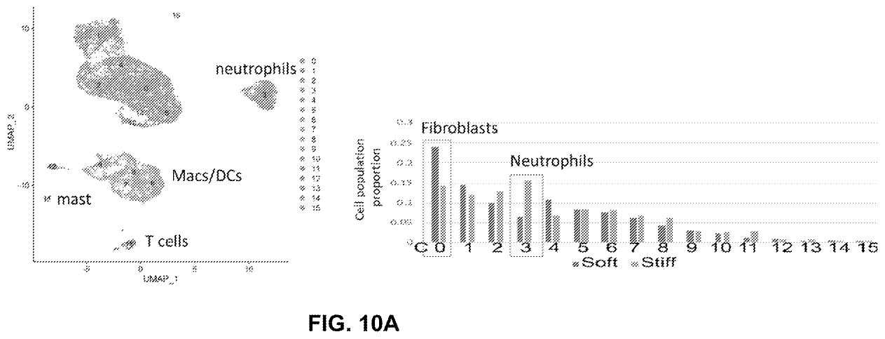

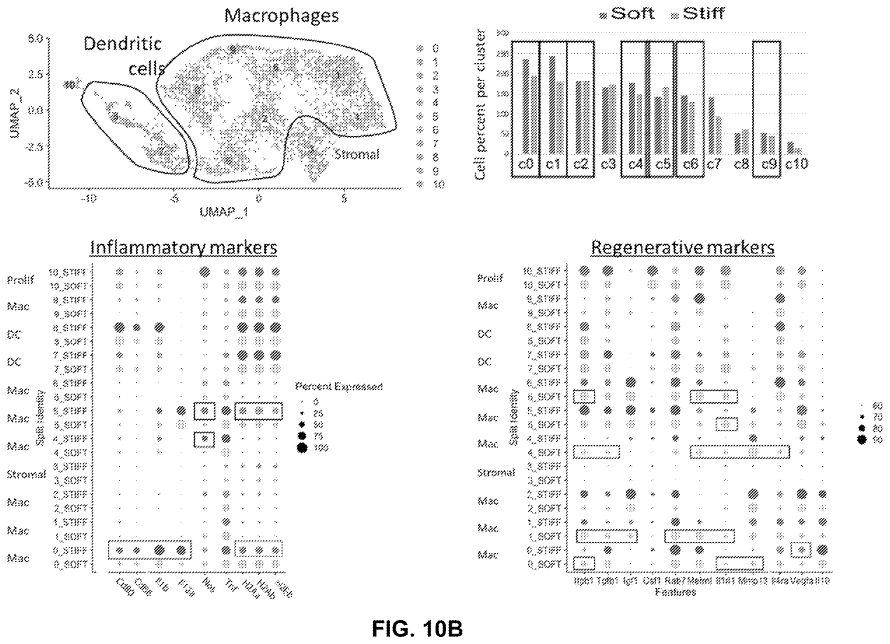

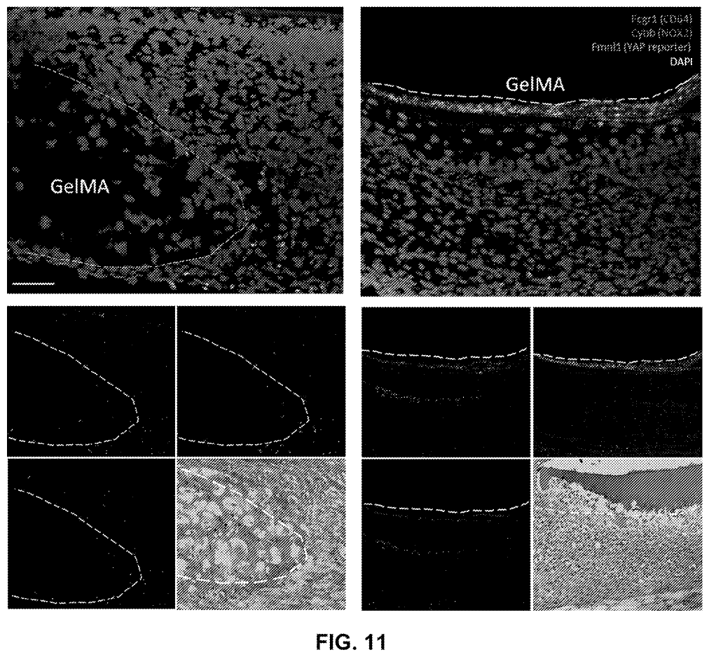

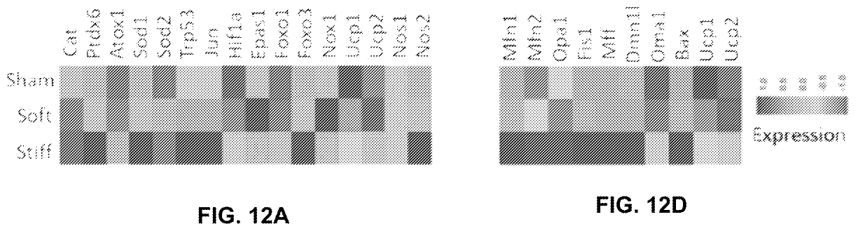

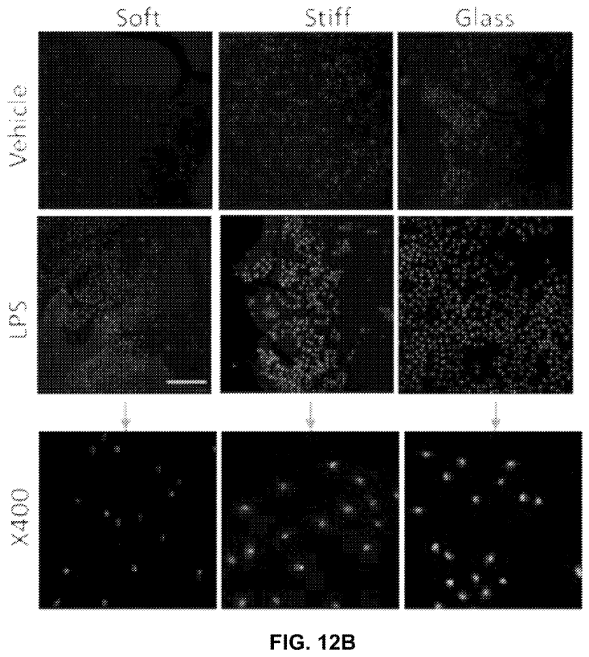

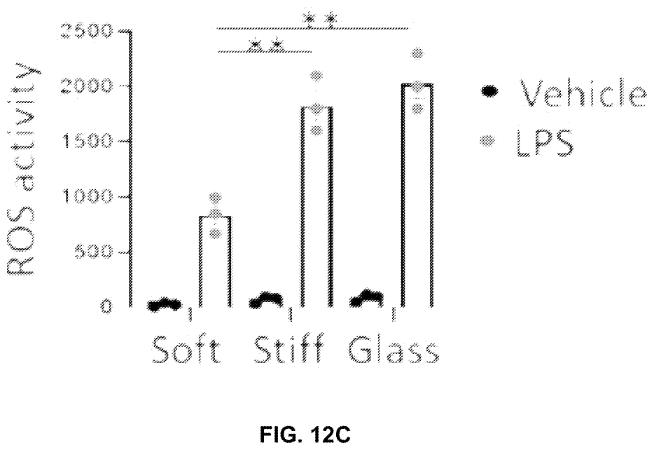

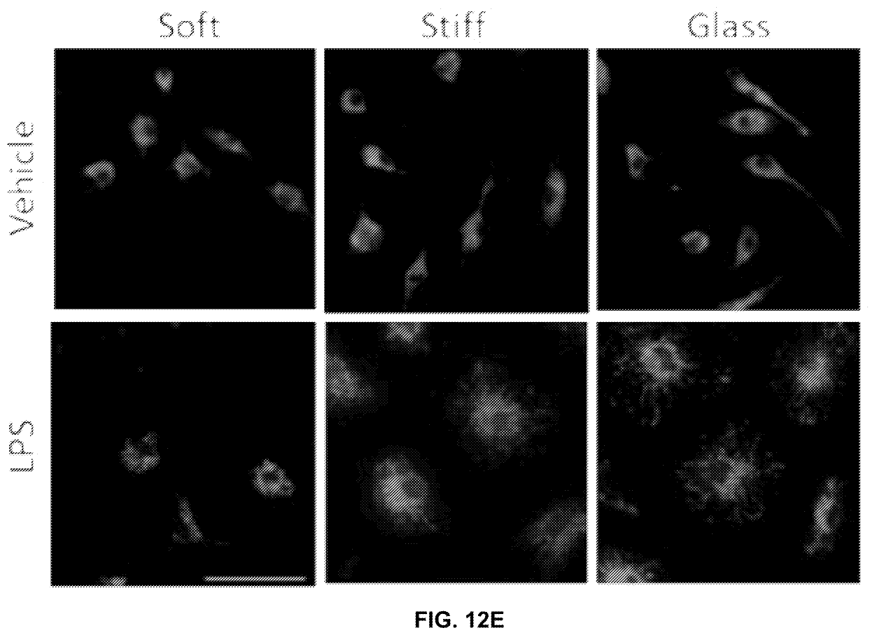

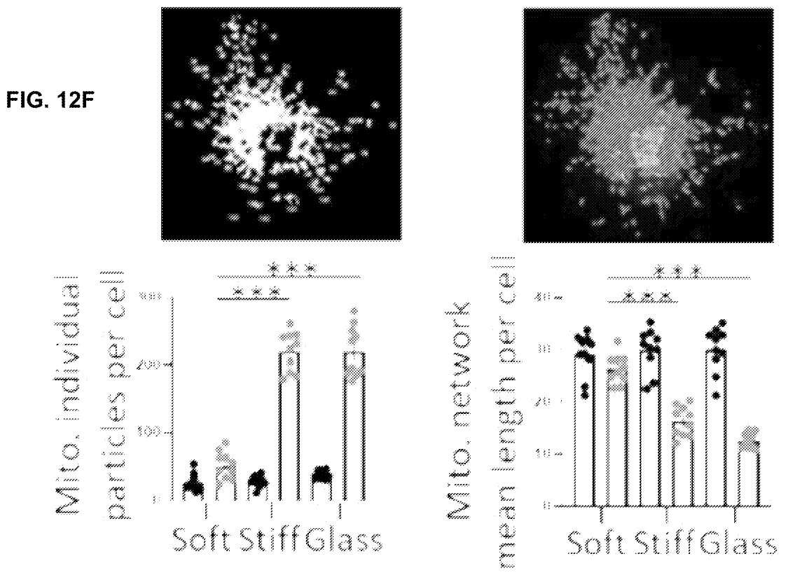

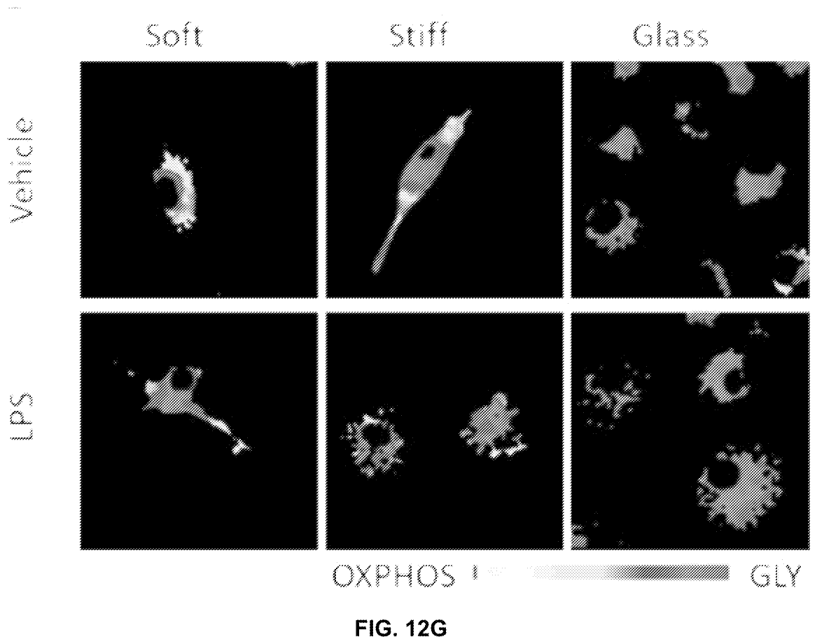

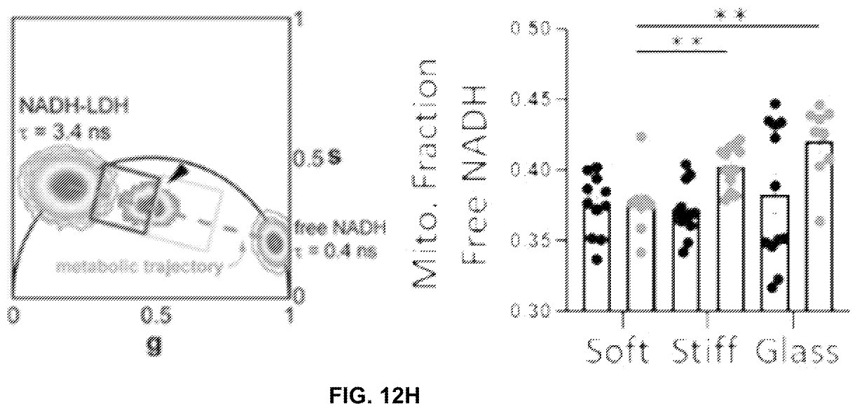

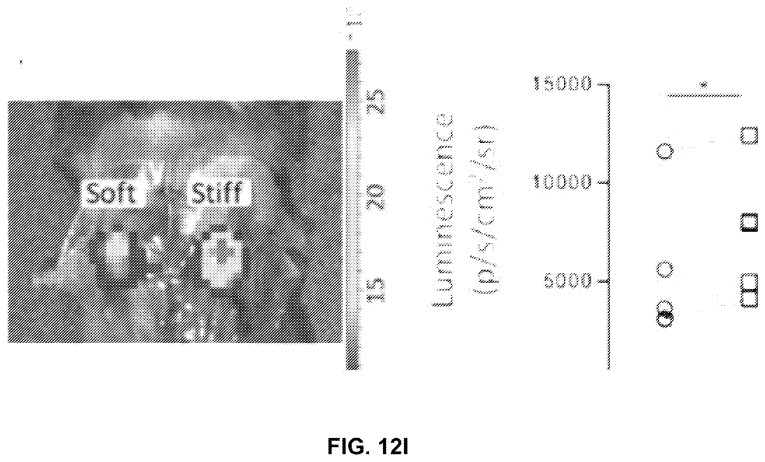

To further compare the immunomodulating effect of gels with stiff or soft properties on various cellular populations in healing wounds, a single-cell RNAseq analysis was performed. Wounds with stiffer GelMA had attracted more neutrophils indicating enhanced inflammation. However, softer GelMA increased the numbers of fibroblasts, indicating enhanced repair activity. Accordingly, stiffer GelMA macrophages presented a stronger inflammatory profile (see FIGS. 10A and 10B). In stiffer gels, macrophages showed stronger activity of several genes related to mechanosensing through YAP. This activity was examined spatially through probing RNA for the expression of the YAP reporter gene—Fmnl. Macrophages that adhere to the stiffer GelMA expressed higher Fmnl, therefore indicating that stiffer gels induce stronger mechanical stimulation together with stronger inflammatory activation (see FIG. 11). Moreover, macrophages in wounds with stiffer gels showed a stronger activity of a group of genes related to reactive oxygen species (ROS) (see FIG. 12A). In accordance with the enhanced inflammation seen with stiff GelMA, these genes implied a ROS-enriched environment in these wounds. To examine the effect of GelMA stiffens on the production of ROS, we cultured bone marrow-derived macrophages (BMDMs) and measured their ROS in the presence of LPS. Stiffer GelMA enhanced the production of ROS by macrophages, indicating that similar might occur in wounds (see FIG. 12B). Moreover, macrophages in wounds with stiffer GelMA showed a stronger activity of genes related to mitochondrial dynamics (see FIG. 12D). Among these genes, both fusion and fission genes were higher in stiff gels, while the central three genes for fission events were highest. To examine mitochondrial fission and fusion, stained were the mitochondrial network on soft and stiff gels in the presence of LPS. It was found that stiffer gels induce mitochondrial fission which corresponds to the mitochondrial phenotype under an acute ROS environment (Jezek, J., K. F. Cooper, and R. Strich, Reactive Oxygen Species and Mitochondrial Dynamics: The Yin and Yang of Mitochondrial Dysfunction and Cancer Progression. Antioxidants (Basel), 2018. 7(1)). Furthermore, to understand the effect of this mechanism on macrophage metabolism, BMDMs were analyzed through NADH Autofluorescence Fluorescence Lifetime Imaging Microscopy (FLIM). FLIM showed that BMDMs on stiff GelMA under LPS stimulation were more glycolytic that the BMDMs on softer GelMA which relied more on oxidative phosphorylation metabolism (see FIG. 12G) To examine the in vivo effect of GelMA stiffness on the ROS burden in wounds, injected were mice with the ROS responsive molecule luminol and imaged the wounds. Wounds with stiffer gels showed enhanced ROS signals (see FIG. 12I). Interestingly, the signal was strong enough for detection mostly on day one post wounding. This indicates that the effect is attributed to neutrophils that constitute the majority of immune infiltration at this early time. To this end, it was seen that stiff GelMA stimulates a stronger inflammatory response in wounds. This is accompanied by enchased production of ROS by neutrophils and macrophages with enhanced macrophage inflammatory activation and neutrophil influx. Additionally, stiff GelMA induces mechanically stimulation of macrophages that enhances inflammatory activation. In conclusion of the mechanical studies, fabricated were GelMA-AN with 10% GelMA and exposure to 1 minute of UV (360 nm).

FIGS. 10A and 10B illustrate example aspects of how stiff GelMA induces more inflammatory activity in key immune cells during wound healing according to embodiments. Single-cell RNAseq shows that: A. Stiffer GelMA increases the numbers of neutrophils indicating enhanced inflammation, while softer GelMA increased the numbers of fibroblasts, indicating enhanced repair activity. B. Stiffer GelMA increases macrophages' inflammatory profile.

FIG. 11 illustrates example aspects of how stiff GelMA stimulates macrophage mechanotransduction according to embodiments. Wounds treated with soft vs. stiff GelMA were stained with an RNA probe against macrophage markers CD64 and Nox2 together with the YAP reporter gene—Fmnl. Macrophages in contact with stiff GelMA show higher Fmnl expression, indicating stronger mechanical stimulation.

FIGS. 12A to 12I illustrate example aspects of how GelMA increased stiffness potentiates macrophage response to LPS by elevating the production of ROS, mitochondrial fission, and glycolysis according to embodiments: A. Expression profile of genes associated with ROS activity in wound macrophages of PWD 5. B. Representative images of BMDMs seeded on GelMA substrates with different stiffness for 24h followed by LPS stimulation for 16 h and stained with CellROX-green indicating ROS production. C. Quantification of CellROX-green staining. D. Expression profile of genes associated with mitochondrial dynamics in wound macrophages of PWD 5. E. Representative images of BMDMs seeded on GelMA substrates with different stiffness for 24 h followed by LPS stimulation for 16 h and stained with MitoTracker Red CMXRos. F. Quantification of individual mitochondrial particles and branched mitochondrial network particles. G. Representative images of BMDMs seeded on GelMA substrates with different stiffness for 24 h followed by LPS followed by imaging with NADH Autofluorescence Fluorescence Lifetime Imaging Microscopy (FLIM). H. Quantification of FLIM signal.

### Example Synthesis of GelMA-AN

Gelatin from porcine skin (Gel) (CAS: 9000-70-8) was dissolved at 10% (w/v) in phosphate-buffered saline (PBS) by stirring at 60 °C using a magnetic stirrer. Methacrylic anhydride (MA) (CAS: 760-93-0) was added dropwise to the gelatin solution to obtain a GelMA solution of 0.8 mL MA per gram of Gel. The reaction mixture was then left under vigorous stirring at 60 °C /3 hrs. Then, the reaction was stopped by a 5-fold dilution of the polymer solution with warm (40 °C) PBS. The resulting solution was dialyzed against deionized water using a 12-14 kDa cut-off dialysis membrane at 40-50 °C for one week while the deionized water was refreshed twice a day. The solution was lyophilized for 1 week to obtain white foam which was stored at −80 °C until further use.

Neutrophil cells were prepared from an 8-10 week old bone marrow from the tibia and femur. Whole bone marrow was flushed out with PBS, spined down, and subjected to red blood cells lysis buffer (ACK Lysing Buffer, ThermoFisher) for 1 minute. Neutrophils were isolated from the bone marrow cells using anti-Ly-6G MicroBeads (Miltenyi Biotec) using the manufacturer's instructions. Isolated neutrophils were rendered apoptotic by incubation in 1% BSA DMEM at 42 °C/1 hr followed by 37 °C/5% CO2/overnight. AN was verified for 100% apoptosis by flow cytometry using Annexin-PI stain (BD). AN were mixed with GelMA.

GelMA was reconstituted in PBS at 50 °C to a concentration of 10% and AN were mixed with GelMA to a concentration of 0.75E6 AN/1ml and Irgacure-2959 (CAS: 106797-53-9) was mixed with GelMA-AN to a final concentration of 0.5%.

Materials derived from natural extracellular matrix have advantages of biocompatibility due to structural and biochemical similarities to the host wound environment. The base scaffold for GelMA-AN is gelatin which is completely biocompatible and FDA approved (Id.). The second part, the AN have been approved to use in clinical trials and are under ongoing investigation for ameliorating inflammatory conditions such as sepsis, arthritis, and other (Id.). We have combined the two components and uncovered a wound healing mechanism that acts through immunomodulation to enhance regenerative healing.

The mechanism works by modulating immune cells to drive them from inflammatory to healing activities that in turn stimulate stromal cells to repair the skin and regenerate healthy skin appendages such as vasculature. This is especially relevant in chronic wounds such as diabetic ulcers but also for other uses like reducing scarring in surgical wounds. Importantly, GelMA-AN is likely to be found beneficial also in non-skin tissues such as muscle. Moreover, we found the specifics in GelMA fabrication that affect the physical properties that modulate immunity. Stiffness of GelMA was found to affect the degree of mechanical sensing of skin immune cells. The degree of crosslinking was found to affect the GelMA accessibility to immune infiltration and degradation. By controlling the degree of crosslinking and stiffness depending on the degree of UV exposure, we found one minute of UV exposure to produce soft GelMA, compared to five minutes of stiff GelMA. Soft GelMA-treated wounds heal with a lower immune response to stiffens which leads to less inflammation and results in smaller scars.

While there are already numerous biomaterial-based scaffolds used clinically, the effects of these materials on the immune cell mediators of healing and regeneration are largely unknown and are still under investigation. Importantly, biomaterials interact with host immune cells, modulating their biochemical and physical environment and downstream activation responses (Kwon, S. H., J. Padmanabhan, and G. C. Gurtner, Chapter 14 Mechanobiology of skin diseases and wound healing, in Mechanobiology in Health and Disease, S. W. Verbruggen, Editor. 2018, Academic Press. p. 415-448; Ozkale, B., M. S. Sakar, and D. J. Mooney, Active biomaterials for mechanobiology. Biomaterials, 2021. 267: p. 120497). Moreover, a few studies have shown that biomaterials can stimulate complete regeneration through mechanisms involving immune modulation (Sun, G., et al., Dextran hydrogel scaffolds enhance angiogenic responses and promote complete skin regeneration during burn wound healing. Proc Natl Acad Sci U S A, 2011. 108(52): p. 20976-81; Griffin, D. R., et al., Activating an adaptive immune response from a hydrogel scaffold imparts regenerative wound healing. Nat. Mater., 2021. 20(4): p. 560-569), suggesting that biomaterial-induced immunomodulation might be a key feature for inducing regenerative healing. GelMA-AN takes advantage of this potential of harnessing immune cells to enhance regenerative healing.

While natural biomaterials have been applied extensively to heal wounds, they also display inherent shortcomings, including the high cost of manufacture and limited flexibility to adjust their biophysical and biochemical properties according to clinical demands. Many of these properties are essential for the successful integration of immunomodulatory biomaterials as a regenerative wound dressing (Gaharwar, A. K., I. Singh, and A. Khademhosseini, Engineered biomaterials for in situ tissue regeneration. Nat. Rev. Mater., 2020. 5(9): p. 686-705). Producing GelMA-AN doesn't involve a high cost. It can be easily produced. It is highly biocompatible and is completely absorbed into the recipient tissue. It has an intriguing mechanism to modulate immune cells in wounds and the healing outcomes of wounds. Among the much more expensive biomaterials for wound healing, there are decellularizing tissues from animals or human cadavers or engineered tissues synthesized from natural ECM components such as fibrin, collagen, and hyaluronic acid. Other common natural components found in wound dressings are polymers of non-mammalian origins such as alginate, chitosan, and dextran.

Cellular-based therapies have largely been unable to alter disease outcomes in regenerative medicine applications (Larson, R. C. and M. V. Maus, Recent advances and discoveries in the mechanisms and functions of CAR T cells. Nat. Rev. Cancer, 2021. 21(3): p. 145-161; Kimbrel, E. A. and R. Lanza, Next-generation stem cells-ushering in a new era of cell-based therapies. Nat. Rev. Drug Discov., 2020. 19(7): p. 463-479; Raffin, C., L. T. Vo, and J. A. Bluestone, Treg cell-based therapies: challenges and perspectives. Nat. Rev. Immunol., 2020. 20(3): p. 158-172). Introducing cells with engineered biomaterials has fared better with modest improvement in wound healing outcomes in specific chronic wound conditions, but also with tremendous cost (Pourmoussa, A., et al., An update and review of cell-based wound dressings and their integration into clinical practice. Ann. Transl. Med., 2016. 4(23): p. 457). GelMA-AN promotes healing at low costs. Importantly, GelMA-AN does it not only cheap but also safe by using the endogenous skin immune cells and skewing their activity without introducing exogenous agents.

While most approaches for skin wound healing have utilized fibroblast and keratinocytes to replenish damaged resident cells, as well as endothelial cells to enhance angiogenesis (Varkey, M., J. Ding, and E. Tredget, Advances in Skin Substitutes—Potential of Tissue Engineered Skin for Facilitating Anti-Fibrotic Healing. Journal of Functional Biomaterials, 2015. 6(3): p. 547-563; Vig, K., et al., Advances in Skin Regeneration Using Tissue Engineering. International Journal of Molecular Sciences, 2017. 18(4): p. 789; Dai, C., S. Shih, and A. Khachemoune, Skin substitutes for acute and chronic wound healing: an updated review. J Dermatolog Treat, 2020. 31(6): p. 639-648), delivery of immune cells or progenitor cells has shown promise for immunomodulation. Studies have shown that delivery of monocytes or anti-fibrotic Trem2+macrophages via pullalan-collagen composite hydrogels could directly skew the immune response toward accelerated wound healing (Hu, M.S., et al., Delivery of monocyte lineage cells in a biomimetic scaffold enhances tissue repair. JCI Insight, 2017. 2(19); Henn, D., et al., Xenogeneic skin transplantation promotes angiogenesis and tissue regeneration through activated Trem2(+) macrophages. Sci Adv, 2021. 7(49): p. eabi 4528). GelMA-AN achieves both stimulations of endogenous immune and stromal cells without involving costly and potentially risky live-cell implementation. By using cells that are already in the process of dying (apoptosis), GelMA-AN is risk-free of tumorgenicity. Furthermore, by using autologous cells for preparing AN (which was not done in mice but can be done in humans), GelMA-AN is risk-free of virulence. These contribute to both the high safety and low cost of GelMA production.

Another cellular component that is studied in biomaterials for wound healing is stem cells. Stem or progenitor cells have the proliferative capacity to expand within tissues, the ability to differentiate into many different cell types, and broad immunomodulatory effects (Gnecchi, M., et al., Paracrine action accounts for marked protection of ischemic heart by Akt-modified mesenchymal stem cells. Nat. Med., 2005. 11(4): p. 367-368; Brennan, M. A., P. Layrolle, and D. J. Mooney, Biomaterials functionalized with MSC secreted extracellular vesicles and soluble factors for tissue regeneration. Adv. Funct. Mater., 2020. 30(37): p. 1909125; Dong, Y., et al., Injectable and tunable gelatin hydrogels enhance stem cell retention and improve cutaneous wound healing. Adv. Funct. Mater., 2017. 27(24): p. 1606619.). Importantly, live-cell implantation holds inherent risks of immune rejection and tumorigenicity. GelMA-AN achieves stimulation of stromal cells, including stem cells, to promote regenerative wound healing without using live cells.

Porosity plays an important role in biomaterials for wound healing because it is necessary to allow for the distribution of diffusible signals, nutrients, and oxygen, especially in the absence of a functional vascular system (Annabi, N., et al., Controlling the porosity and microarchitecture of hydrogels for tissue engineering. Tissue Eng. Part B Rev., 2010. 16(4): p. 371-383). These promote cell infiltration and new tissue formation Porosity can also be engineered and approaches to introduce porosity include the use of porogens, solvent casting and particulate leaching, lyophilization, or freezing (cryogels) (Op ′t Veld, R.C., et al., Design considerations for hydrogel wound dressings: Strategic and molecular advances. Tissue Eng. Part B Rev., 2020. 26(3): p. 230-248; Woeller, C. F., et al., Evaluating a variable porosity wound dressing with anti-scar properties in a porcine model of wound healing. Eplasty, 2018. 18: p. e 20.). GelMA-AN porosity is highly tunable by several factors that modify the gelatin scaffold and also by the amount of AN intercalated in the gel. The gelatin scaffold is tunable by a combination of the following factor: First, the incorporation of methacrylate to provide functional groups for crosslinking and the degree of methacrylation determines the final crosslinked structure. The amount of photoinitiator and duration of UV exposure also determines the final crosslinked structure. In GelMA-AN, we found the right combination of the above (see above) to form a porous hydrogel that stimulates the infiltration of cells indicative of both accessibility and diffusible signaling.

As mentioned above, GelMA-AN enriches the healing wounds with growth and angiogenic factors by stimulating their production by endogenous macrophages (see FIG. 4). Same growth factors, such as FGF and EGF, are used in the treatment of chronic wounds (Id.). Moreover, GelMA-AN also enriches the healing wounds with growth factors that have demonstrated efficacy for surgical use to enhance healing and reduce scarring including PDGF, VEGF, and TGF-β (Id.). However, instead of costly recombinant protein production, GelMA-AN achieves the same in a cost-efficient way (see FIG. 4). The GelMA-AN mechanism works in a way that produces the effect on the immune cells and the production of growth factors gradually and over time. This action is obtained because of the continuous interaction of the GelMA-AN with the immune system which includes penetration into GelMA-AN, its breakdown, and production of the healing effect. This mechanism is much more effective than acute delivery of a bioactive molecule that might either insufficient or too high a dose that produces side effects. Additionally, because GelMA-AN stimulates the production of growth factors from endogenous cells it is safer as it does not pose a risk of high doses and repeated applications. Importantly, AN additive prolongs GelMA-AN persistence in wounds compared to GelMA alone. This not only contributes to additional pro-healing effects but also prolongs the pro-angiogenic effect of the hydrogels which results in increased vasculature (see FIGS. 2A-2E). This is likely to be a result of an increased phagocytic burden on the macrophages that delays the degradation of hydrogels

The present Applicants have performed work on a murine model of wound healing to study GelMA-AN. Created was a 5 mm full excisional punch wound. The wounds are splinted with a silicon ring and sutures to mimic the healing model of human's tight skin by minimizing the contraction of mice's panniculus carnosus in response to wounding. In these wounds, it is observed that GelMA-AN which is prepared specifically as described above is stimulating significant angiogenesis in wounds at post-wounding day 14.

Furthermore, assessed are the GelMA-AN effect on macrophage cells in vitro. There it is found that GelMA-AN reduces their inflammatory stimulation in response to bacterial mimetics LPS. Moreover, it is found that GelMA and AN both stimulate macrophage production of anti-inflammatory and pro-repair growth factors. Each component stimulates a different profile of these growth factors but in their combined form, GelMA-AN achieves complementary production.

### Example Aims in Accordance With Embodiments

Impaired wound healing in response to injury, surgery, or during disease is a major clinical problem, and new treatment strategies to improve tissue repair are needed. In the skin, wound healing is initiated by blood coagulation and inflammation, followed by tissue remodeling and scar formation. An essential component of this process is angiogenesis, the formation of new blood vessels to promote tissue oxygenation as well as infiltration of additional immune cells to facilitate healing. However, inadequate angiogenesis, particularly in diabetic patients or healing of large wounds, is a major hurdle and results in slowed healing and worsened scar. Delivery of angiogenic growth factors (e.g. platelet derived growth factor or PDGF and vascular endothelial growth factor or VEGF) and cells (e.g. endothelial cells) have been attempted, but without clinical success. Thus, new strategies to promote angiogenesis during wound healing are desired.

Embodiments provide a new therapeutic approach that leverages natural interactions of immune cells within wounds to stimulate angiogenesis. Specifically, embodiments deliver autologous apoptotic neutrophils (AN), which will potently induce angiogenesis signaling in local macrophages). Neutrophils are the first leukocytes to arrive at sites of injury, phagocytosing and eliminating microorganisms and damaged cells, and releasing reactive species, serine and matrix metalloproteinases, and proteases. Following these activities, most neutrophils will readily undergo programmed cell death, or anontosis This is then followed by monocyte infiltration and differentiation to macrophages (referred to herein as Mφs), which engulf and clear AN present within the wound, a process termed efferocytosis. Efferocytosis of AN by Mos coincides with a dramatic shift in the wound environment, where inflammation begins to subside and healing progresses with increased angiogenesis and tissue remodeling. Thus, it is anticipated that exogenous delivery of AN will potently stimulate Mφs to undergo a phenotypic transition, enhance angiogenesis and wound healing, without causing superfluous inflammation.

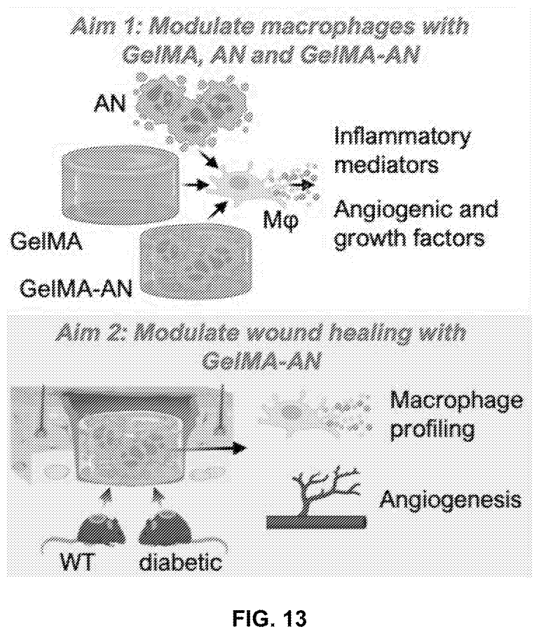

The present Applicant has found that delivery of AN in a degradable gelatin methacrylate (GelMA)-based hydrogel profoundly enhanced the extent of angiogenesis in full thickness splinted murine skin wounds. Strikingly, blood vessels were induced not only in wounds of wildtype (WT), but also diabetic db/db mice, which typically have scarce vasculature. Moreover, culture of Mos with AN and/or GelMA enhanced their secretion of angiogenic growth factors including PDGF and VEGF, as well as the anti-inflammatory cytokine IL-10. Thus, it is considered that delivery of AN will promote angiogenesis and mitigate inflammation through direct interactions with Mos. This is explored by investigating the effects of neutrophils and degradable natural biomaterials on Mo uptake and growth factor secretion (in Aim 1) and angiogenesis and wound healing of full thickness skin wounds (in Aim 2). Such a project significantly advances knowledge of AN-and biomaterial-induced angiogenesis and wound healing through the following example Specific Aims (see FIG. 13):

Specific Aim 1: Consider that GelMA-AN promotes angiogenesis and anti-inflammatory signaling in Mo. Examples will characterize the effects of AN generated by different apoptosisinducing methods (temperature, drugs, UV) and an array of natural biomaterials on Mo uptake and secretion of angiogenic growth factors and inflammatory cytokines. These and other examples will encapsulate AN within biomaterials to examine their combined effects on Mo uptake and function. In addition, examples will examine the effects of media conditioned by Mos treated with GelMA and/or AN on angiogenesis in vitro.

Specific Aim 2: Consider that GelMA-AN enhances angiogenesis and healing of acute skin wounds through modulation of Mo. Examples apply GelMA-AN to skin wounds of healthy and diabetic mice, and characterize cellular infiltrate by histology and immunohistochemistry (IHC). Blood vessels will be evaluated by IHC and 3D imaging techniques. Flow cytometry and single cell RNA sequencing will characterize Mon subpopulations and angiogenic signaling, and spatial transcriptomics will be used to evaluate proximity of subpopulations to the hydrogel in situ. Finally, examples will study the role of Mφs by depleting Mos and evaluating the angiogenesis response to GelMA-AN.

IMPACT: This high-risk high-reward project will establish that delivery of AN in a hydrogel enhances Mo angiogenesis signaling. This work will provide a platform to further investigate mechanistic signals underlying these responses and develop a platform for therapeutic intervention leveraging AN, autologous cell therapy, for improved wound healing.

SIGNIFICANCE: Impaired wound healing is a major clinical challenge.

Every year over 6 million patients suffer from impaired wound healing resulting from pathological events, traumatic injuries, and surgical procedures. (Eming, S. A., Martin, P. & Tomic-Canic, M. Wound repair and regeneration: mechanisms, signaling, and translation. Science translational medicine 6, 265sr 266, doi: 10.1126/scitranslmed.3009337 (2014); Sen, C. K. et al. Human skin wounds: a major and snowballing threat to public health and the economy. Wound Repair Regen 17, 763-771, doi: 10.1111/j.1524-75X.2009.00543.x (2009)) Burns, acute and chronic wounds, as well as many skin diseases often manifest in a collagen-rich scar tissue. Diabetic patients in particular exhibit impaired healing, with delayed healing and worsened scar. Tissue healing that results in scar can lead to organ failure and is a huge burden on public health and healthcare costs. (Eming, S. A., Krieg, T. & Davidson, J. M. Inflammation in wound repair: molecular and cellular mechanisms. The Journal of investigative dermatology 127, 514-525, doi: 10.1038/sj.jid.5700701 (2007)).

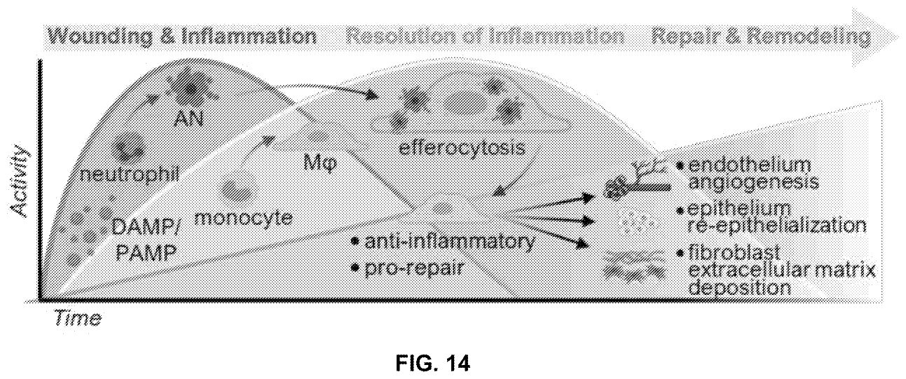

Role of immune cells and angiogenesis wound healing: The first immune cells recruited to wounds-mostly neutrophils-function to phagocytose and eliminate damaged cells and foreign micro-organisms (see FIG. 14). Sites of neutrophil activity generate a cytotoxic environment through oxidative burst and release of proteases and neutrophil extracellular traps. A by-product of this exuberant killing is neutrophils undergoing programmed cell death, or apoptosis. (Filep, J. G. & Ariel, A. Neutrophil heterogeneity and fate in inflamed tissues: implications for the resolution of inflammation. American Journal of Physiology-Cell Physiology 319, C510-C532 (2020)). Concurrently, monocyte influx into the tissue and mature into Mos, engulfing AN in a non-inflammatory process termed efferocytosis. This clearance is a critical checkpoint that triggers resolution of inflammation by blocking neutrophil recruitment, eliminating early inflammatory components and driving successful wound healing. (Doran, A. C., Yurdagul, A. & Tabas, I. Efferocytosis in health and disease. Nature Reviews Immunology 20, 254-267, doi: 10.1038/s41577-019-0240-6 (2020).) Efferocytosis not only removes AN, but also induces an anti-inflammatory and pro-repair Mφ phenotype that is critical for successful healing. Mφ reprogramming is characterized by suppression of pro-inflammatory cytokines such as TNF and IL-12 along with increased production of pro-resolving cytokines such as IL-10. (Satyanarayanan, S. K. et al. IFN-β is a macrophage-derived effector cytokine facilitating the resolution of bacterial inflammation. Nature Communications 10, 1-16, doi:10.1038/s41467-019-10903-9 (2019); Butenko, S. et al. Transcriptomic Analysis of Monocyte-Derived Non-Phagocytic Macrophages Favors a Role in Limiting Tissue Repair and Fibrosis. Frontiers in Immunology 11, 405, doi: 10.3389/fimmu.2020.00405 (2020); Elliott, M. R., Koster, K. M. & Murphy, P. S. Efferocytosis Signaling in the Regulation of Macrophage Inflammatory Responses. J Immunol 198, 1387-1394, doi:10.4049/jimmunol.1601520(2017).) Mφ anti-inflammatory activation is closely tied to pro-repair activation that is characterized by the production of growth and angiogenic factors such as vascular endothelial growth factor (VEGF), platelet derived growth factor (PDGF), and transforming growth factor-b (TGFb) (Wynn, T. A. & Vannella, K. M. Macrophages in Tissue Repair, Regeneration, and Fibrosis. Immunity 44, 450-462, doi: 10.1016/j. immuni.2016.02.015 (2016)). This is necessary for resolution of inflammation and healthy healing of tissue and defects in this stage often give rise to non-healing wounds (Phillipson, M. & Kubes, P. The Healing Power of Neutrophils. Trends in Immunology 40, 635-647, doi: 10.1016/j.it.2019.05.001 (2019)).

A major component of the provisional matrix is the formation of new capillary networks to aid in the further influx of leukocytes and provide oxygenation to the newly healed tissue. Early wound Mo interact with blood vessel tips, driving sprouting by acting as a local source of VEGFA. (Gurevich, D. B. et al. Live imaging of wound angiogenesis reveals macrophage orchestrated vessel sprouting and regression. EMBO J 37,doi:10.15252/embj.201797786 (2018)). At later stages, Mo drive vasculature maturation by producing TGFb, PDGF, and fibroblast growth factor (FGF) (Id.). Interestingly, several approaches have attempted to deliver angiogenic growth factors for wound treatment or post-surgical scar mitigation. (Park, J. W., Hwang, S. R. & Yoon, I. S. Advanced Growth Factor Delivery Systems in Wound Management and Skin Regeneration. Molecules 22, doi:10.3390/molecules22081259 (2017); Abdelhakim, M., Lin, X. & Ogawa, R The Japanese Experience with Basic Fibroblast Growth Factor in Cutaneous Wound Management and Scar Prevention: A Systematic Review of Clinical and Biological Aspects. Dermatol Ther (Heidelb) 10, 569-587, doi: 10.1007/s13555-020-00407-6 (2020); Trengove, N. J., Bielefeldt-Ohmann, H. & Stacey, M. C. Mitogenic activity and cytokine levels in nonhealing and healing chronic leg ulcers. Wound Repair Regen 8, 13-25, doi:10.1046/j.1524-475x.2000.00013.x (2000); Krzyszczyk, P., Schloss, R., Palmer, A. & Berthiaume, F. The Role of Macrophages in Acute and Chronic Wound Healing and Interventions to Promote Pro-wound Healing Phenotypes. Front Physiol 9, 419, doi:10.3389/fphys.2018.00419 (2018)).

Cell-and biomaterial-based therapies for wound healing. Immunomodulatory approaches have recently emerged as a powerful strategy for wound treatment, including biomaterials that regulate immune cell activities by interacting with them directly. Natural biomaterials such as fibrin, collagen, and hyaluronic acid generally have beneficial effects, have anti-inflammatory and pro-healing effects in many wound healing models. (Butenko, S. et al. Engineering Immunomodulatory Biomaterials to Drive Skin Wounds toward Regenerative Healing. Cold Spring Harb Perspect Biol, doi:10.1101/cshperspect. a041242 (2022).) Cell delivery approaches include myeloid cells to modulate the immune environment18, fibroblasts for dermal repair (Pourmoussa, A., Gardner, D. J., Johnson, M. B. & Wong, A. K. An update and review of cell-based wound dressings and their integration into clinical practice. Ann. Transl. Med. 4, 457, doi: 10.21037/atm.2016.12.44 (2016); Dai, C., Shih, S. & Khachemoune, A. Skin substitutes for acute and chronic wound healing: an updated review. J Dermatolog Treat 31, 639-648, doi: 10.1080/09546634.2018.1530443 (2020)) or endothelial or stem cells to promote angiogenesis. However, their safety and prolonged effects and survival remains a major challenge. Embodiments provide a new approach that delivers AN short-term, since they are designed to be cleared by host immune system, coaxing a reprogramming of host Mo that, in turn, subsides inflammation and enhances signaling to aid in wound healing. Furthermore, biomaterial crosslinking is engineered to control its degradation and host cell access to AN. It is considered that together, this biomaterial-AN platform will shift the wound environment to promote angiogenesis, matrix deposition, and better healing.

INNOVATION: Embodiments provide a completely novel therapeutic approach to improve wound healing that leverages natural interactions between innate immune cells that ultimately stimulates angiogenesis. The present embodiments: (1) unveil new knowledge about how Mo interactions with AN regulate angiogenesis and inflammatory signaling, and (2) develop a new approach to treat skin wounds. Embodiments include the following conceptual innovations:

New knowledge about how AN influence the function of Mos. The present work is the first to demonstrate that AN embedded in a hydrogel stimulate angiogenesis and anti-inflammatory signaling in Mos. The present embodiments unveil whether the method of inducing apoptosis, type of apoptic cells, and direct interactions and/or engulfment are necessary for this response.

New knowledge about how and direct interactions and/or engulfment are necessary for this response. interactions with biomaterials influence their angiogenic and antinflammatory potential. The present embodiments examine the role of matrix biomaterial recognition, binding and uptake in Mo cytokine and growth factor secretion.

New strategies to improve angiogenesis during wound healing. A better understanding of how AN and hydrogels may reveal new therapeutic approaches for improved healing.

In addition, embodiments leverage the following technical innovations:

Engineered biomaterial platform for delivery of apoptotic cell bodies. Embodiments develop crosslinked natural biomaterials that serve as delivery platforms for apoptotic cells into wounded tissues. The effects of crosslinking on cell delivery efficacy and immune response will be optimized.

Spatial transcriptomic profiling to evaluate Mo phenotypes in situ. Embodiments utilize spatial transcriptomics to examine the responses of wound cells directly within the tissue. Proximity to AN and hydrogel scaffolds will help determine whether interactions are needed for functional responses.

EXAMPLE PRELIMINARY WORK: Apoptotic neutrophils (AN) delivered via a gelatin methacrylate (GelMA) scaffold induces angiogenesis in splinted full thickness skin wounds of WT and db/db mice: To investigate the effect of GelMA-AN on wound healing, created were two 5 mm full-thickness excision punch wounds on the dorsal skin of each mouse, using both wild-type (WT) and homozygous diabetic (Leprdb or db/db) mice. The wounds were splinted with a sutured silicone ring to prevent contraction and received GelMA, GelMA-AN, or sham treatments.

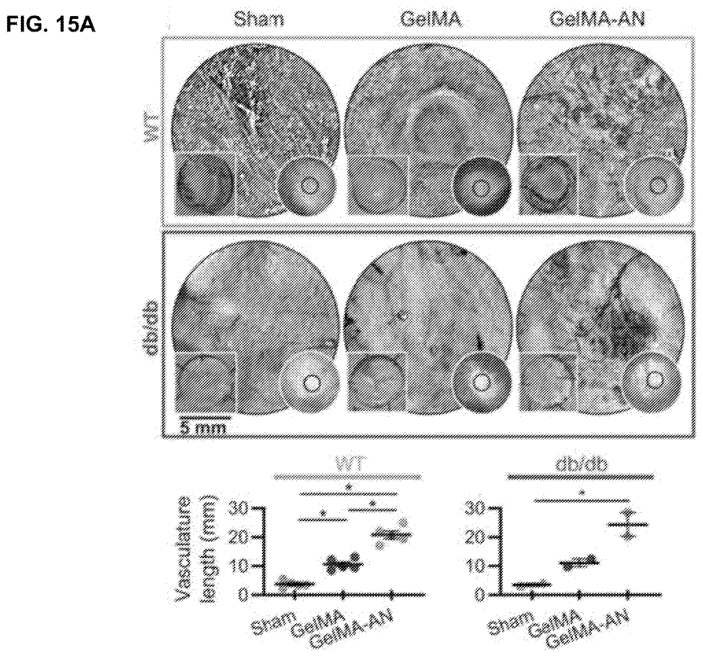

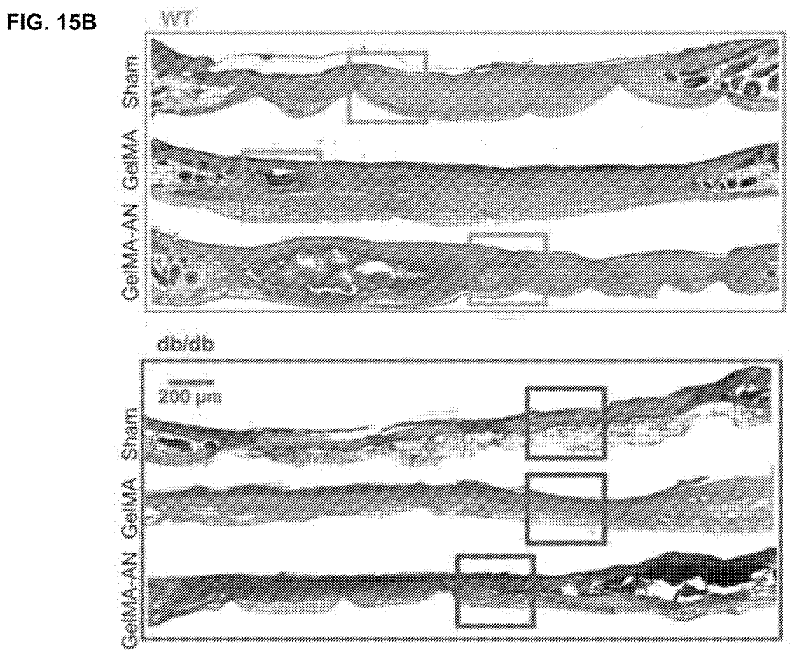

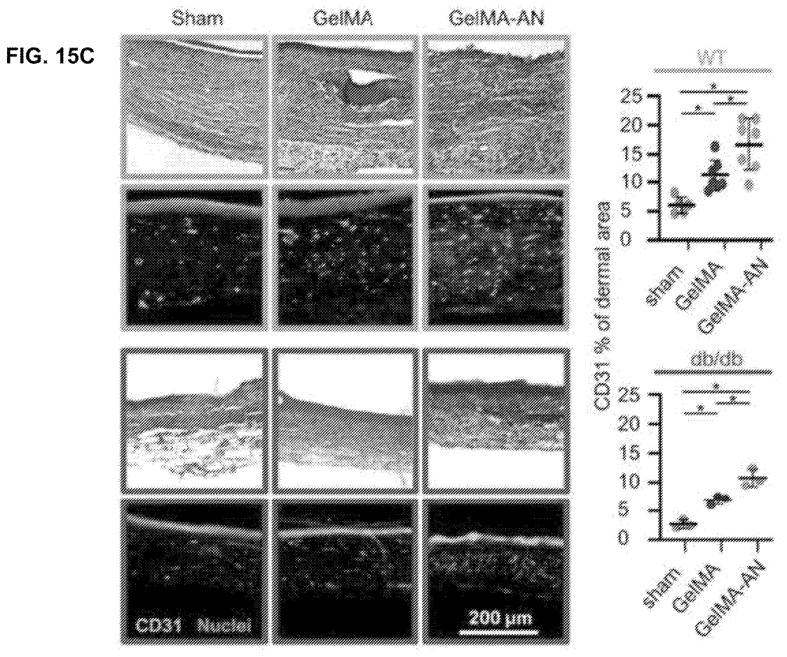

All wounds were covered with Telfa and Tegaderm and allowed to heal for 14 d, after which they were examined using a dissection microscope, histology, and immunohistochemistry. Observed was significantly enhanced vasculature in WT mice treated with GelMA and GelMA-AN compared to sham (n=5-7 female mice for each condition). Additionally, GelMA-AN showed a more significant increase in vasculature than GelMA alone (see FIGS. 15A-15B). Moreover, GelMA and GelMA-AN prolonged immune infiltration with higher cellular numbers in wounds at PWD14 (see FIG. 15B). These trends repeated in preliminary work with db/db mice (n=2-3 female mice), and additional studies in more mice of both sexes will verify these results in our proposed work. Together, these data suggest that GelMA-AN enhances the pro-healing properties of GelMA in wound healing, likely through interactions with immune cells.

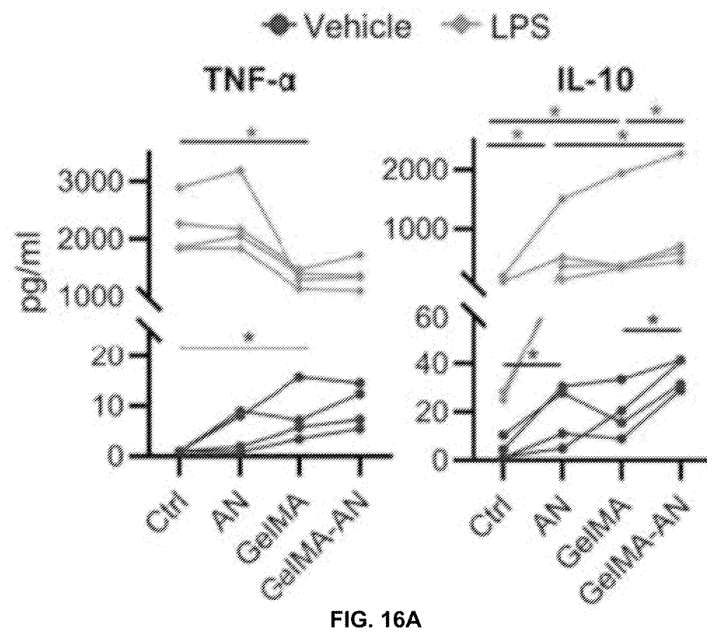

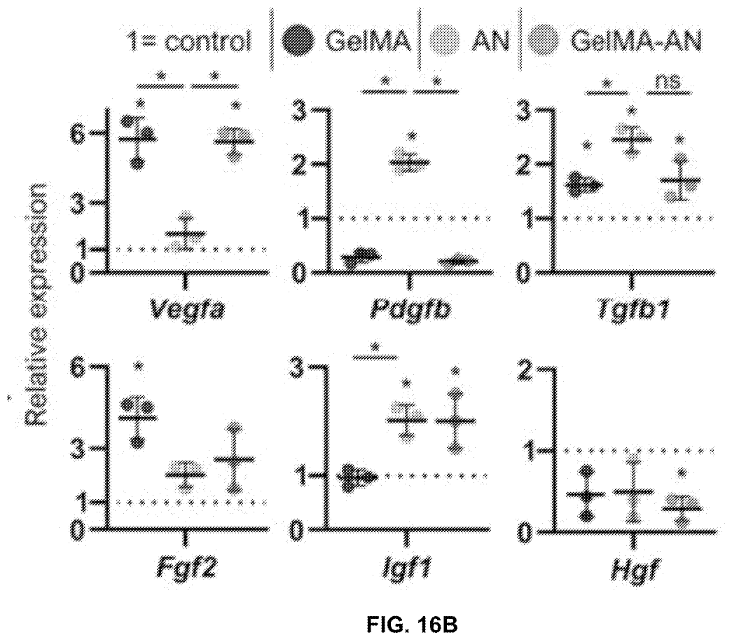

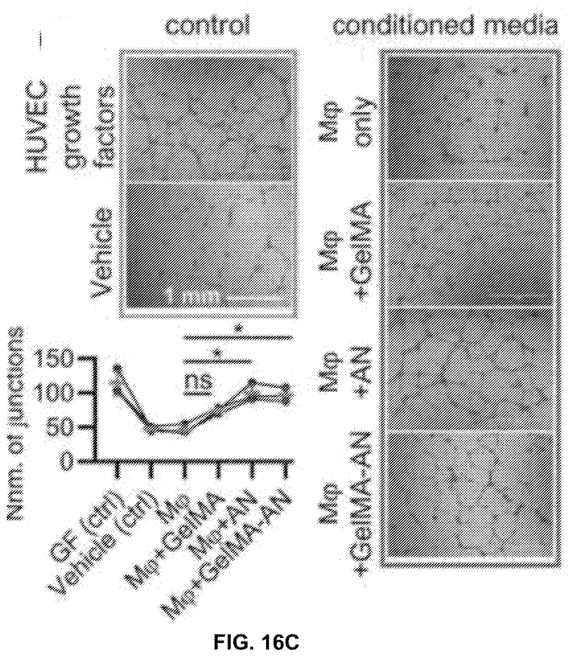

AN and GelMA reduce inflammatory cytokines and stimulate anti-inflammatory cytokines and angiogenic growth factor in Mo: To investigate the immunomodulatory effects of GelMA and GelMA-AN, we cultured bone marrow-derived Mφs (BMDMs), stimulated them with AN, GelMA, or GelMA-AN and analyzed the activated inflammatory TNFa (see FIG. 15B). However, when Mos were stimulated with LPS, GelMA inhibited the secretion of TNFa while enhancing the secretion of anti-inflammatory IL-10. AN also increased IL-10, and GelMA-AN further enhanced IL-10 in a synergistic manner (see FIG. 16A). These data suggest that GelMA and AN combination can shift Mφs towards anti-inflammatory and pro-healing state. GelMA also has the potential to provide protection from inflammatory activation.

FIGS. 15A to 15C illustrate example Angiogenic potential of GelMA-AN according to embodiments: (A) Dissection microscope images of PWD14 wound show GelMA-AN stimulates vascularization. (B) Small images: splinted wounds (left) whole wound diameter (right) (C) Histological analysis. H&E; showing dense immune infiltrate with GelMA and GelMA-AN, dense concentration of blood vessels with GelMA-AN (blue arrows). CD31; Showing significantly enhanced staining with GelMA-AN. * p<0.05 by ANOVA.

To evaluate the effect of GelMA-AN on growth and angiogenic factor production in BMDM, qPCR is performed and it was found that Vegfa, Tgfb1, Fgf2, Igf1 were all upregulated by GelMA and/or AN (see FIG. 16B). Pdgfb was upregulated only by AN and downregulated by GelMA containing conditions, and Hgf was downregulated by all treatments, with GelMA-AN showing a significant effect.

Finally, to functionally evaluate the angiogenic potential, performed was a human umbilical vein endothelial cell (HUVEC) tube formation assay with conditioned media from Mφ treated with GelMA, AN, and GelMA-AN. It was found that conditioned media from AN and GelMA-AN-treated Mo enhanced HUVEC tube formation (FIG. 16C). Overall, these data suggest that GelMA and AN have a positive and often complementary effect on growth and angiogenic factor production by Mφs.

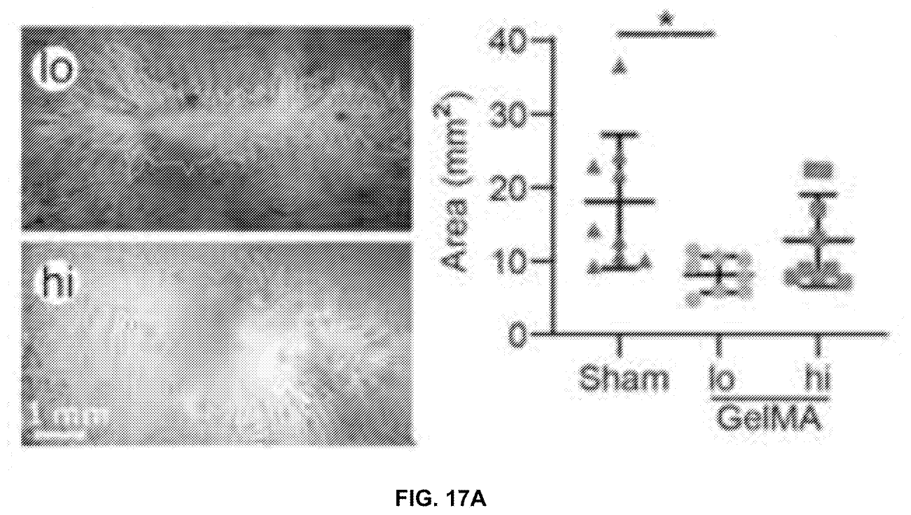

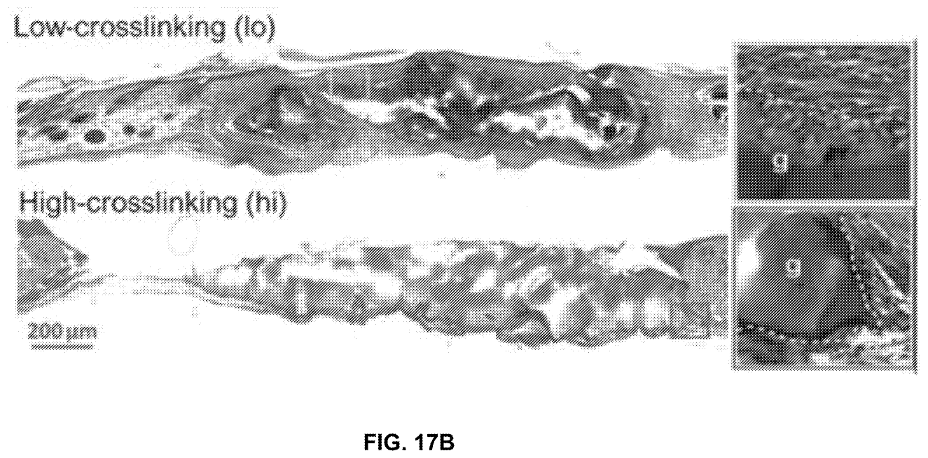

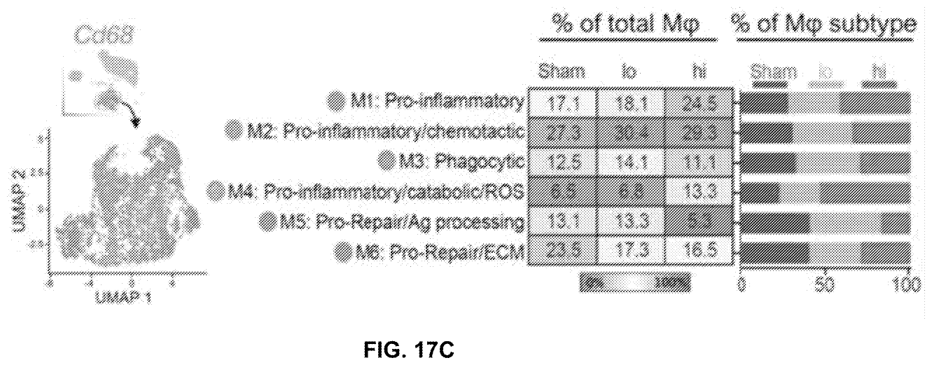

GelMA crosslinking modulates interactions with Mφ and the repair response: To investigate the effects of hydrogel crosslinking on the healing full-thickness skin wounds, UV-crosslinking was used to create lightly crosslinked and soft GelMA hydrogels (lo-GelMA; 3 kPa) and heavily crosslinked and stiff hydrogels (hi-GelMA; 150kPa) and applied them to 5 mm full thickness murine skin wounds. At PWD-30, both lo-and hi-GelMA showed a trend towards smaller scar size compared to untreated wounds, with lo-GelMA showing a significant reduction (see FIG. 17A). H&E staining showed that at PWD-5, lo-GelMA began to integrate into the skin, whereas, hi-GelMA showed minimal cell infiltration (see FIG. 17B). To investigate the impact of crosslinking on wound healing, used was scRNAseq to study the entire wound cell infiltrate. Focusing on the Cd68^{high}/Mrc1^{high} Mφ subpopulations, we identified six functional subpopulations (M1-6) based on up-regulated genes and GO pathway analysis (see FIG. 17C). Clusters M1 and M4 are upregulated by hi-GelMA, and exhibited pro-inflammatory and oxidative signatures. In contrast, M3 and M5 are upregulated by lo-GelMA and show phagocytic and prorepair activities. These findings suggest that GelMA crosslinking impacts the Mo functional state, potentially through changes in degradation and phagocytic uptake. In other embodiments, it is anticipated that AN encapsulated within a crosslinked hydrogel will promote pro-angiogenic effects on Mφ, as long as the extent of crosslinking allows access to AN. Degradation and uptake of the hydrogel itself by Mφ may also enhance reparative processes, and separate and synergistic effects of AN efferocytosis and hydrogel phagocytosis will be tested.

Summary and Rigor of Example Prior and Preliminary Work: Embodiments provide a new strategy to promote angiogenesis during wound healing using an immunomodulatory biomaterial that promotes sustained Mo production of growth factors. Although there is significant prior work considering biomaterial-and cell-based therapies no studies have explored the potential of delivery AN within hydrogels to treat wounds. Our preliminary data suggest that GelMA-AN induces significant increase in vasculature compared to GelMA alone, and enhances growth and angiogenic factors production. GelMA and AN also skew Mo towards anti-inflammatory and pro-healing activation. The effects of hydrogel crosslinking on the macrophage response in full-thickness skin wounds were also investigated, with lo-GelMA showing a significant reduction in scar area than hi-GelMA. Preliminary experiments described above were performed across at least five animals (except studies in db/db animals), and other experiments will increase the sample size in both sexes. These findings suggest that GelMA-AN has potential in enhancing the pro-healing properties of GelMA in wound healing.

FIGS. 16A to 16C illustrate example synergistic effects of GelMA-AN on Mφ polarization and angiogenesis according to embodiments: (A) ELISA of TNFa and IL-10 secreted by Mo treated with GelMA and/or ANs. (B) qPCR of angiogenesis and growth factor expression by Mφ treated with GelMA and/or AN. (C) HUVEC tube formation is enhanced by conditioned media from Mo treated with AN and GelMA-AN. * p<0.05 by ANOVA.

FIGS. 17A to 17C illustrate example GelMA crosslinking modulates Mφ phenotype and scarring of wounds according to embodiments: (A) Whole mounted wounds at PWD30 show reduced scaring with lo-GelMA. Yellow dashed line is scar outline. Scale 1 mm. n=10 per group. * p<0.05 by ANOVA. (B) H&E staining of PWD 5 sections indicating incorporation and degradation of lo-GelMA vs. bulk hi-GelMA Scale 200 um. Yellow square: lo-GelMA: cell infiltration into GelMA. Red square: hi-GelMA: cell alignment on GelMA edge. Yellow dashed line: GelMA-dermis border. (C) scRNAseq showing monocyte-Mφ-GelMA are enriched for pro-inflammatory (M1) and oxidative (M4) subpopulation and lo-GelMA are enriched for pro-healing (M5) and phagocytic (M3) subpopulations.

Example Specific Aim 1: Test the hypothesis that AN and gelatin methacrylate (GelMA) promote angiogenesis signaling and reduce inflammation in Mos.

Rationale: AN release signals that promote their efferocytosis by Mφs, modulating inflammation and tissue repair. (Elliott, M. R., Koster, K. M. & Murphy, P. S. Efferocytosis signaling in the regulation of macrophage inflammatory responses. The Journal of Immunology 198, 1387-1394 (2017).) Our preliminary work suggests that culture of Mos with AN and/or GelMA induces angiogenesis growth factor and reduces inflammatory cytokine production. In addition, it is found in related work that other natural biomaterial matrices including collagen and fibrin also reduce the inflammatory activation of Mφs. (Meli, V. S. et al. YAP-mediated mechanotransduction tunes the macrophage inflammatory response. Sci. Adv. 6, eabb8471 (2020); Hsieh, J. Y. et al. Matrix crosslinking enhances macrophage adhesion, migration, and inflammatory activation. APL Bioeng 3, 016103, doi:10.1063/1.5067301(2019 ).) In example Aim 1, examples test the following factors in our AN-hydrogel platform: (1) method of inducing cell death (2) cell type (neutrophils vs. other), (3) direct contact and/or engulfment of dead cells (3) hydrogel type (GelMA vs. other), and (4) the extent of hydrogel crosslinking. These studies will advance understanding of Mos angiogenic signaling induced by apoptotic cells and/or hydrogels, for optimization of angiogenesis-inducing wound dressing.

Example Cell source: Embodiments use bone marrow derived Mφs (BMDM) from C57BL/6J mice, which have been widely used in studies of Mφs and comprise a substantial proportion of Mφs recruited to wounds. Cells will be harvested and differentiated as described previously. (McWhorter, F. Y., Wang, T., Nguyen, P., Chung, T. & Liu, W. F. Modulation of macrophage phenotype by cell shape. Proc Natl Acad Sci U S A 110, 17253-17258, doi:10.1073/pnas.1308887110 (2013); Wang, T., Luu, T. U., Chen, A., Khine, M. & Liu, W. F. Topographical modulation of macrophage phenotype by shrink-film multi-scale wrinkles. Biomater Sci 4, 948-952, doi: 10.1039/c6bm00224b (2016).) Neutrophils will be obtained by isolating Ly 6g+ neutrophils from bone marrow via magnetic antibody. 7-12 week old male and female mice will be used to ensure mature immune systems. Selected studies will also be performed in macrophages from db/db mice, since their macrophages may be dysfunctional (Khanna, S. et al. Macrophage dysfunction impairs resolution of inflammation in the wounds of diabetic mice. PLoS One 5, e9539, doi:10.1371/journal. pone.0009539 (2010)) and human peripheral blood neutrophils and mononuclear cellderived Mos. Donor blood will be obtained from UCI Institute for Clinical and Translational Sciences (ICTS) Normal Blood Program, as done previously. (Chen, E. Y. et al. CD200 modulates macrophage cytokine secretion and phagocytosis in response to poly(lactic co-glycolic acid) microparticles and films. Journal of materials chemistry. B 5, 1574-1584, doi:10.1039/c6tb02269c (2017 ).)

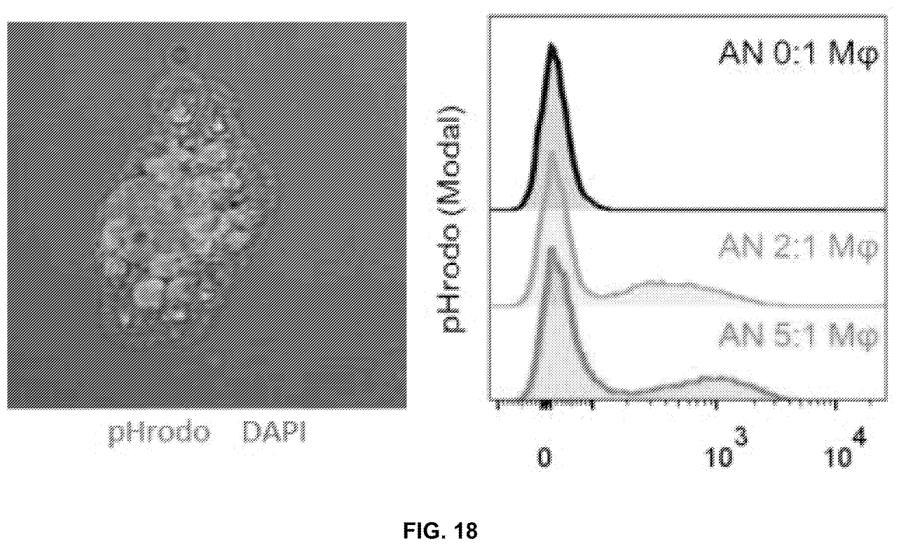

Experimental design: Aim 1.1 Characterize the effects of AN on Mo uptake and angiogenic signaling. Neutrophil apoptosis will be induced by incubating at 42 °C for 1 h (as in example preliminary work), staurosporine or ionomycin treatment6, and UV irradiation (Id.); followed by 37 °C overnight incubation. As controls, examples will induce necrosis by freeze/thaw cycle or ethanol treatment and use other cell types such as fibroblasts (NIH 3T3) and thyomcytes (isolated from murine thymus). (Zhen, Y. & Shao, W. H. Experimental Analysis of Apoptotic Thymocyte Engulfment by Macrophages. J Vis Exp, doi:10.3791/59731(2019 ).) Dead cells will be labelled with pHrodo (succinimidyl ester, which fluoresces upon acidification in the phagolysosome, Invitrogen), applied to Mφs for 24 h, and uptake will be evaluated by microscopy and/or flow cytometry (see FIG. 18).

Cytokines and growth factors will be examined by qPCR and ELISA. To identify potentially new molecular regulators of AN-induced responses, examples will perform bulk RNA sequencing (RNA-seq) of Mos cultured with apoptotic cells (one type chosen with highest angiogenesis signals) and evaluate differential genes upon treatment.

To verify that direct interactions and/or uptake are needed for Mφ responses to GelMA and AN, examples will use conditioned media, transwell assays, and pharmacological inhibitors to block efferocytosis and phagocytosis. Supernatant from AN will be collected and transferred to Mos cultures or AN will be co-cultured with Mφ while separated by a transwell membrane. In addition, Mφs will be incubated with GelMA-AN along with phagocytosis or efferocytosis inhibitors (cytochalasin D, bafilomycin, Bavituximab, Annexin A5; available from ThermoFisher).

Finally, to functionally evaluate the impact of Mφ secreted products on angiogenesis, supernatants from will be collected and added to human umbilical vein endothelial cells (HUVECs) and murine 3B-11 endothelial cells (both commonly used in angiogenesis studies (DeCicco-Skinner, K. L. et al. Endothelial cell tube formation assay for the in vitro study of angiogenesis. J Vis Exp, e51312, doi:10.3791/51312(2014 ); Vidal, R. et al. Transcriptional heterogeneity of fibroblasts is a hallmark of the aging heart. JCI Insight 4, doi:10.1172/jci.insight.131092 (2019)) cultured on Matrigel-coated surfaces 30 or a 3D fibrin bead angiogenesis assay. (Welch-Reardon, K. M. et al. Angiogenic sprouting is regulated by endothelial cell expression of Slug. J Cell Sci 127, 2017-2028, doi:10.1242/jcs.143420 (2014)). Preliminary work performed with Dr. Alan Widgerow (see letter) show prominent changes in network formation with Mo conditioned media (FIG. 3C).

Aim 1.2. Investigate the effects of biomaterial scaffolds on Mφ uptake and angiogenic signaling. In addition to GelMA used in preliminary work, exmaples will fabricate other matrix hydrogels including fibrin, collagen, and hyaluronic acid (available from EMD Millipore, Advanced Biomatrix), as well as synthetic biomaterials such as polyethylene glycol (PEG, Creative PEGWorks) that lack cell adhesion sites, all of which can be crosslinked to different extents. (Hsieh, J. Y. et al. Matrix crosslinking enhances macrophage adhesion, migration, and inflammatory activation. APL Bioengineering 3, 10.1063/1061.5067301(2019 ); Hu, H. & Xu, F. J. Rational design and latest advances of polysaccharide-based hydrogels for wound healing. Biomater Sci, doi:10.1039/d0bm00055h (2020); Sallent, I., Capella-Monsonis, H. & Zeugolis, D. I. Production and Characterization of Chemically Cross-Linked Collagen Scaffolds. Methods in molecular biology (Clifton, N.J.) 1944, 23-38, doi:10.1007/978-1-4939-9095-5_3 (2019); Yamauchi, M., Terajima, M. & Shiiba, M. Lysine Hydroxylation and Cross-Linking of Collagen. Methods in molecular biology (Clifton, N.J.) 1934, 309-324, doi:10.1007/978-1-4939-9055-9_19 (2019).) Mφs will be cultured on various hydrogels, with high and low crosslinking, and cytokine and growth factor secretion will be measured as we have previously done (Meli, V. S. et al. YAP-mediated mechanotransduction tunes the macrophage inflammatory response. Sci. Adv. 6, eabb8471 (2020); Hsieh, J. Y. et al. Matrix crosslinking enhances macrophage adhesion, migration, and inflammatory activation. APL Bioeng 3, 016103, doi:10.1063/1.5067301 (2019)) and in preliminary work (FIG. 3A). To measure hydrogel uptake, pHRodo will be added to hydrogels, BMDMs seeded on the labeled gel will produce a fluorescent signal once the gel is engulfed into the phagolysosome acidic environment (FIG. 6). Uptake and functional responses will be evaluated as in Aim 1.1.

FIG. 18 illustrates example Mo uptake of labeled AN according to embodiments: BMDMs cultured for 4 h with pHrodo (red)-labeled AN and measured by confocal (left) microscopy and flow cytometry (right).

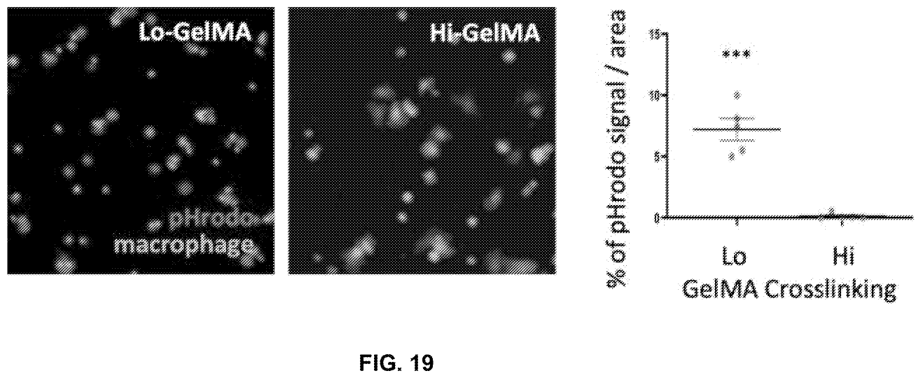

FIG. 19 illustrates example Crosslinking influences GelMA uptake by Mφs according to embodiments. Fluorescent images (left) and quantification (right) of rhodamine signal in Mos cultured on pHrodo-labelled GelMA, with high and low crosslinking. ***p<0.001 by t-test.

Aim 1.3: Determine the combined effects of apoptotic cells and biomaterials on Mφ uptake and angiogenic signaling. Apoptotic cells and biomaterials determined to have the greatest pro-angiogenetic/anti-inflammatory effect will be combined and tested for uptake and Mφ functional response. Cells and hydrogel will be fluorescently labeled with two different fluorophores, and uptake measured a described above. Effects of hydrogel crosslinking on AN/biomaterial uptake and Mo inflammation and angiogenic signaling will be evaluated.

Expected outcomes, pitfalls and alternatives for Aim 1: Example studies strongly suggest that GelMA-AN reduces inflammatory cytokine and induces angiogenic growth factor production by Mos. This Aim will provide critical information about whether the method of inducing apoptosis or the type of cell and biomaterial used for delivery impacts the Mφ response. It is anticipated that the method of inducing apoptosis may not cause significantly different responses, but necrotic cells may be more inflammatory. Other cell types may also have muted effects. If it is determined in Aim 1.1 that a soluble factor from AN is sufficient, Aim 1.3 will be modified to use AN supernatant. In addition, while example studies will focus on angiogenic effects mediated by Mφs, it is also possible that AN-GelMA will influence ECM deposition and fibroblasts. If H&E staining in Aim 2 reveals significant changes in matrix architecture, examples will additionally examine the effects on fibroblasts and ECM deposition.

Power Analysis and Statistical Plan for Aim 1: Assays will be performed with duplicate samples in at least 5 biological replicates (cells from different mice or donors). Differences caused by AN or biomaterials will be evaluated by t-test or ANOVA and Tukey's posthoc test. In some cases, where variability due to mouse-to-mouse or donor-to-donor differences are too high, normalization and paired statistical tests will be performed.

Example Specific Aim 2: Test the hypothesis that GelMA-AN enhances angiogenesis and healing of acute skin wounds through modulation of Mφs. Rationale: To probe whether GelMA-AN influences Mφs, angiogenesis, and wound healing in vivo, we will apply GelMA-AN dressings to full thickness splinted wounds. Mice have been chosen for their relatively short time for wound closure, allowing study of the entire healing cascade. Embodiments evaluate Mos dynamics and new blood vessels with histology, immunohistochemistry (IHC) and flow cytometry. To comprehensively analyze markers associated with complex Mφ phenotypes found in vivo, which often do not overlap with in vitro markers, single cell RNA-sequencing pipeline developed by co-I Plikus will be used. (Guerrero-Juarez, C. F. et al. Single-cell analysis reveals fibroblast heterogeneity and myeloid-derived adipocyte progenitors in murine skin wounds. Nature communications 10,650, doi:10.1038/s41467-018-08247-x (2019); Gay, D. et al. Phagocytosis of Wnt inhibitor SFRP4 by late wound macrophages drives chronic Wnt activity for fibrotic skin healing. Science Advances (In Press)).

Animal model and ethical statement: Experiments will be performed on C57B1/6J wild type, diabetic (db/db) and endothelium reporter Tie1/2-GFP mice (all available from Jackson Laboratory). To eliminate the effect of hair cycle stage differences (Stojadinovic, O., Ito, M. & Tomic-Canic, M. in J Invest Dermatol Vol. 131(2)292-294(2011); Ansell, D., Kloepper, J., Thomason, H., Paus, R. & Hardman, M. Exploring the “hair growth-wound healing connection”: anagen phase promotes wound re-epithelialization. J Invest Dermatol 131, 518-528, doi:10.1038/jid.2010.291 (2011)), all wounds will be created in 50 d old animals, when HFs are predictably in the resting state (telogen). Two 5 mm full thickness excisional wounds will be created in the intrascapular dorsal skin region in shaved mice and a splint will be sutured around the wound to prevent contraction. Dressings (or saline control) will be applied immediately after wounding, photo-crosslinked as described in our previous work (Hsieh, J. Y. et al. Matrix crosslinking enhances macrophage adhesion, migration, and inflammatory activation. APL Bioengineering 3, 10.1063/1061.5067301 (2019)), and covered with Tegaderm (3M). Male and female mice will be analyzed separately to account for sex-specific differences. (Rønø, B., Engelholm, L., Lund, L. & Hald, A. Gender affects skin wound healing in plasminogen deficient mice. PLOS ONE 8, e59942, doi:10.1371/journal. pone.0059942 (2013); Santos, J. & Monte-Alto-Costa, A. Female, but not male, mice show delayed cutaneous wound healing following aspirin administration. Clinical and Experimental . . . , doi:10.1111/1440-1681.12043 (2013); Campbell, L., Emmerson, E. & Davies . . . , F. Estrogen promotes cutaneous wound healing via estrogen receptor β independent of its antiinflammatory activities. The Journal of . . . (2010).) All studies will be conducted according to the UCI IACUC guidelines.

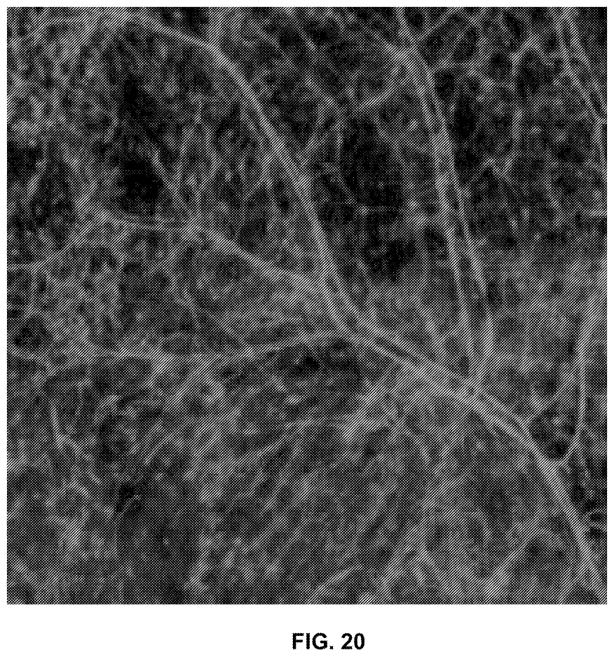

Experimental design: Aim 2.1: Study the effect of GelMA-AN on angiogenesis in healthy and diabetic mouse wounds by histology and immunohistochemistry. Examples will evaluate the effects of GelMA-AN, GelMA, and no treatment controls over a time course of healing. Final scar size will be evaluated at 30 d by morphometric analysis (as performed in FIGS. 17A-18C). At 5, 14, and 30 d after wounding and treatment, animals will be sacrificed, and wounds will be analyzed by histology and IHC (Table 1). H&E will be used to assess inflammation (expected at 5 d) and vascularization (expected at 14 and 30 d). IHC will be used to quantify neutrophils (MPO), Mφs (F4/80), and endothelial cells (CD31) We will quantify the proportion of inflammatory or wound healing Mφs using CD86 (inflammation) or CD206 (healing). To further analyze blood vessel networks, we will utilize Tie1/2-GFP mice (available from Jackson), in which endothelial cells are labelled with GFP, and analyze cleared tissues at 14 d after wounding (see FIG. 20). Examples will quantify the number and area of blood vessels by morphometry using ImageJ (NIH).

Table 1: Study Design for Aim 2.1.

FIG. 20 illustrates example Tie1/2GFP imaged at 15 d post wounding according to embodiments.

Aim 2.2: Analyze Mφ subpopulations using flow cytometry and single cell RNA sequencing (scRNAseq). Mo subpopulations will be quantified by FACS, which will require pooling of samples from 3 wounds for each biological replicate. Comparisons will be made between no treatment, GelMA and GelMA-AN hydrogels. Examples will study Mφs as the percentage of total cells surrounding the implant and the proportion of healing to inflammatory cells. Mφs will be labeled as F4/80+CD11b+; pro-healing will be distinguished from proinflammatory by CD206hi/CD86low. As an unbiased approach to examine all cell populations and to identify additional Mo subpopulations, examples will perform scRNA-seq. Preliminary analysis of myeloid populations (identified by CD68 and Mrc1) within PWD5 skin wounds treated with GelMA injury reveal at least 6 distinct clusters (e.g. FIGS. 17A-17C), with shifts in population in response to high and low crosslinking. Embodiments similarly analyze cellular infiltrate surrounding GelMA-AN, in order detect numerous Mo subpopulations, which is not possible with FACS or IHC described above. Pathways involving phagocytosis, inflammation, angiogenic signaling will be evaluated. Other cell populations including neutrophils and fibroblasts will be similarly evaluated.

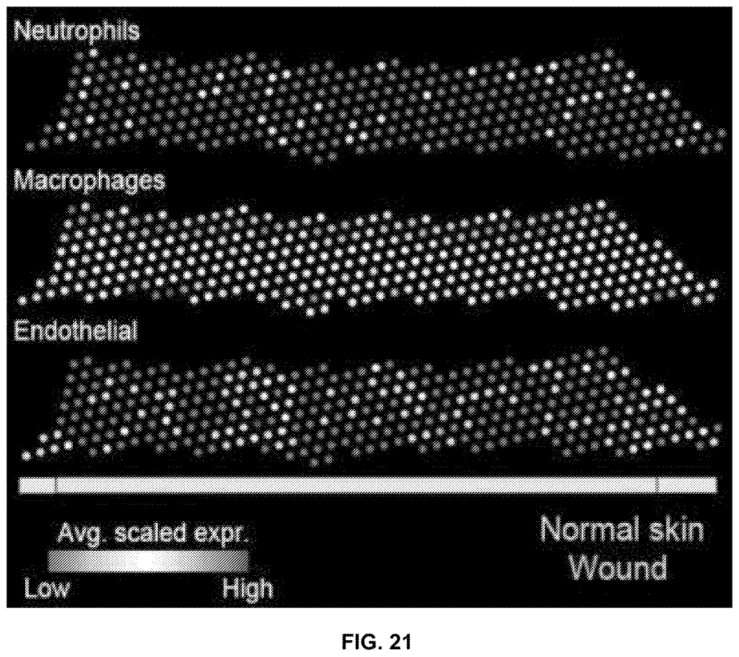

To probe the spatial localization of different Moφ subtypes, examples will utilize spatial transcriptomics techniques recently developed in PI Plikus' lab. Preliminary work in PWD13 murine skin wounds was performed using Visium (10X Genomics). Composite gene scores for neutrophils, Mφ, and endothelial cells superimposed on digitized wound section reveal abundance of Mφ throughout the wound, heterogenous dispersion of endothelial cells and few neutrophils (see FIG. 21). Examples will use this technique to probe different Mo subpopulations and their proximity to the hydrogel and endothelial cells.

Aim 2.3: Investigate the role of Mφ s on GelMA-AN-induced angiogenesis. To test whether Mφs are required for GelMA-AN induced angiogenesis, examples will deplete Mφs with clodronate liposomes, which will be administered one day prior to wounding. Analysis of healing by histology/IHC will be performed as described in Aim 2.1 at 5 d (to evaluate Mφ) and 14 d (to evaluate vascularization).

Expected outcomes, pitfalls, and alternatives for Aim 2: It is expected to gain insight on how GelMA influences Mo phenotypes and vascularization. If examples identify alternative methods in Aim 1, this Aim will be modified accordingly. It is anticipated that AN will be filtered out during processing and its RNA will not interfere with genomic studies. Studies using clodronate will confirm a role for Mφs, but if few differences are observed, examples will utilize other depletion models (e.g. Rag mice). Phagocytosis blockers described in Aim 1 can also be delivered via GelMA to determine their role in angiogenic signaling. Finally, while example studies focus on angiogenesis and Mφ, other examples may also examine effects on re-epithelialization, fibroblasts, and matrix deposition.

Power Analysis and Statistical Plan for Aim 2: Based on the comparison of previously published work (Wang, T., Luu, T. U., Chen, A., Khine, M. & Liu, W. F. Topographical modulation of macrophage phenotype by shrink-film multi-scale wrinkles. Biomater Sci 4, 948-952, doi:10.1039/c6bm00224b(2016 )), a sample size of 6 mice/sex (per group per time point) will obtain 80% power to detect this observed difference with significance level at 0.05 (Aim 2.1 and 2.3); cells from three animals will be combined for one replicate, and FC and scRNA-seq will be run in triplicate. One or two-way ANOVA followed by Tukey's post-hoc test will be used to evaluate the effect of GelMA-AN, and effects in control vs. depleted models.

Rigor and Reproducibility: Statistical Rigor: Experimental designs and sample sizes are all statistically driven, and determined based on power analysis using mean, standard deviation, and effect size obtained from previous work. Statistical design and analysis of all data will be with the assistance of statisticians UCI's Biostatistics Epidemiology & Research Design (BERD) Unit. Related work provides evidence of performing proposed procedures and assays including (1) bioengineered systems to manipulate Mφ (Luu, T. U., Gott, S. C., Woo, B. W., Rao, M. P. & Liu, W. F. Micro- and Nanopatterned Topographical Cues for Regulating Macrophage Cell Shape and Phenotype.

ACS Appl Mater Interfaces 7, 28665-28672, doi:10.1021/acsami.5b10589 (2015); Liu, W. F., Nelson, C. M., Tan, J. L. & Chen, C. S. Cadherins, RhoA, and Rac1 are differentially required for stretch-mediated proliferation in endothelial versus smooth muscle cells Circulation research 101, e44-52, doi:10.1161/circresaha.107.158329 (2007)) (2) in vitro models to assess of Mo adhesion, phenotype and function (Smith, T. D., Tse, M. J., Read, E. L. & Liu, W. F. Regulation of macrophage polarization and plasticity by complex activation signals. Integrative biology: quantitative biosciences from nano to macro 8, 946-955, doi:10.1039/c6ib00105j (2016); Kim, Y. K., Que, R., Wang, S. W. & Liu, W. F. Modification of biomaterials with a self-protein inhibits the macrophage response. Advanced healthcare materials 3, 989-994, doi:10.1002/adhm.201300532 (2014); Kim, Y. K. et al. Incorporation of a Ligand Peptide for Immune Inhibitory Receptor LAIR-1 on Biomaterial Surfaces Inhibits Macrophage Inflammatory Responses. Adv Healthc Mater 6, doi:10.1002/adhm.201700707 (2017)), (3) murine wound healing. (Wang, Q. et al. A multi-scale model for hair follicles reveals heterogeneous domains driving rapid spatiotemporal hair growth patterning. eLife 6, doi:10.7554/eLife.22772 (2017); Guerrero-Juarez, C. F. et al. Wound Regeneration Deficit in Rats Correlates with Low Morphogenetic Potential and Distinct Transcriptome Profile of Epidermis. The Journal of investigative dermatology 138, 1409-1419, doi:10.1016/j.jid.2017.12.030 (2018); Plikus, M. V. et al. Cyclic dermal BMP signalling regulates stem cell activation during hair regeneration. Nature 451, 340-344, doi:10.1038/nature06457 (2008); Plikus, M. V. et al. Self-organizing and stochastic behaviors during the regeneration of hair stem cells. Science (New York, N.Y.) 332, 586-589,doi:10.1126/science.1201647 (2011); Chen, C. C. et al. Organ-level quorum sensing directs regeneration in hair stem cell populations. Cell 161, 277-290, doi: 10.1016/j. cell.2015.02.016(2015)). Biological variables: (1) Mφs from different organisms vary in their response. Examples will carry out in vitro experiments using cells from 5 mice/experiment. (2) Wound healing among individual mice is variable, and examples base sample sizes on previously determined effect sizes. Males and females will be evaluated separately to account for potential differences caused in gender.

Aim 2 studies may be initiated with current GelMA-AN formulation and may be modified based on results from Aim 1. Completion of the proposed study will provide a platform to (1) study molecular mechanisms underlying GelMA-AN-induced antiogenic signaling in Mφ, (2) develop novel platforms for regenerating other tissues that require improved angiogenesis including the muscle and heart, and (3) further determine the pre-clinical efficacy of GelMA-AN in wound healing and translation of the therapeutic strategy into the clinic.

### Table 2: Study Design for Aim 2.2

FIG. 21 illustrates example spatial transcriptomics of murine 13 d skin wounds according to embodiments. Composite gene scores average scaled expression for neutrophils (S100a8, S100a9, Ly6g), macrophages (Lyz2, Cd68) and endothelial (Pecam1, Tiel, Cdh5) are superimposed on wound sections.

### Table 3: Study Design for Aim 2.3

The herein described subject matter sometimes illustrates different components contained within, or connected with, different other components. It is to be understood that such depicted architectures are illustrative, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected,” or “operably coupled,” to each other to achieve the desired functionality, and any two components capable of being so associated can also be viewed as being “operably coupleable,” to each other to achieve the desired functionality. Specific examples of operably coupleable include but are not limited to physically mateable and/or physically interacting components and/or wirelessly interactable and/or wirelessly interacting components and/or logically interacting and/or logically interactable components.

With respect to the use of plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.).

Although the figures and description may illustrate a specific order of method steps, the order of such steps may differ from what is depicted and described, unless specified differently above. Also, two or more steps may be performed concurrently or with partial concurrence, unless specified differently above. Such variation may depend, for example, on the software and hardware systems chosen and on designer choice. All such variations are within the scope of the disclosure. Likewise, software implementations of the described methods could be accomplished with standard programming techniques with rule-based logic and other logic to accomplish the various connection steps, processing steps, comparison steps, and decision steps.

It will be further understood by those within the art that if a specific number of an introduced claim recitation is intended, such an intent will be explicitly recited in the claim, and in the absence of such recitation, no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations).

Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general, such a construction is intended in the sense one having skill in the art would understand the convention (e.g., “a system having at least one of A, B, or C” would include but not be limited to systems that have A alone, B alone, C alone, A and B together A and C. together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

Further, unless otherwise noted, the use of the words “approximate,” “about,” “around,” “substantially,” etc., mean plus or minus ten percent.

Although the present embodiments have been particularly described with reference to preferred examples thereof, it should be readily apparent to those of ordinary skill in the art that changes and modifications in the form and details may be made without departing from the spirit and scope of the present disclosure. It is intended that the appended claims encompass such changes and modifications.

1. A biomaterial comprising:
immunomodulating properties engineered for complete incorporation into injured tissues while enhancing the regenerative healing activities of immune and stromal cells; and
a neutrophil.

2. The biomaterial of claim 1, wherein the neutrophil is an apoptic neutrophil.

3. The biomaterial of claim 1, wherein a source of the neutrophil is an autologous source or human cell lines.

4. The biomaterial of claim 1, wherein the biomaterial comprises a degradable biomaterial.

5. The biomaterial of claim 4, wherein the degradable biomaterial comprises a matrix that delivers the neutrophil.

6. The biomaterial of claim 5, wherein the matrix comprises one or more of gelatin methacryloyl (GelMA) and a fibrin.

7. The biomaterial of claim, wherein the degradable biomaterial includes one or more of natural extracellular matrix based materials or synthetic biomaterials engineered to contain immunomodulatory and/or cell adhesive and degradable domains.

8. The biomaterial of claim, wherein the degradable biomaterial includes a gelatin component that is supplemented with methacrylic anhydride-forming a cross-linkable biomaterial-gelatin methacryloyl (GelMA).

9. The biomaterial of claim 8, wherein the GelMA is supplemented with a photoinitiator that allows tunable GelMA polymerization in wounds using UV light.

10. The biomaterial of claim 8, wherein the GelMA is a scaffold comprising 10% of GelMA and 90% physiological solution.

11. The biomaterial of claim 8, wherein the GelMA part is mixed with apoptotic neutrophil cells (AN) and forms GelMA-AN.

12. The biomaterial of claim 11, wherein the AN provides immunomodulating components that act on central immune coordinators—macrophage cells.

13. The biomaterial of claim 1, wherein the injured tissues comprise skin or muscle.

14. The biomaterial of claim 1, wherein the biomaterial improves healing by stimulating the infiltration of immune cells and activating them in a way that inhibits inflammation and promotes tissue repair.

15. A method of making a biomaterial comprising:
preparing gelatin from porcine skin (Gel);
dissolving the Gel in a saline;
adding methacrylic anhydride (MA) to the Gel solution to obtain a GelMA solution;
preparing neutrophil cells from bone marrow cells to obtain AN; and
mixing the GelMA solution with the AN to obtain GelMA-AN with a final concentration of about 0.5%.
